(19) United States
(12) Patent Application Publication
(10) Pub. No.: US 2025/0002631 A1
(43) Pub. Date: Jan. 2, 2025

(54) Radically Polymerizable Materials For The Production Of Dental Molding With Little Discoloration

(71) Applicants: Ivoclar Vivadent AG, Schaan (LI); Technische Universität Wien, Wien (AT)

(72) Inventors: Kai RIST, Feldkirch (AT); Norbert MOSZNER, Triesen (LI); Yohann CATEL, Sevelen (CH); Patrick KNAACK, Wien (AT); Robert LISKA, Schleinbach (AT); David BASSENHEIM, Vöslau (AT)

(21) Appl. No.: 18/757,360

(22) Filed: Jun. 27, 2024

(30) Foreign Application Priority Data
Jun. 26, 2023 (EP) 23182872.4

(51) Int. Cl. C08F 290/14; A61C 13/08; A61K 6/15; A61K 6/79; A61K 6/893; C08F 2/50; C08K 5/13

(52) U.S. Cl. CPC C08F 290/147; A61C 13/081; A61K 6/15; A61K 6/79; A61K 6/893; C08F 2/50; C08K 5/13; C08K 2201/012

(57) ABSTRACT

A free radical polymerization initiator system having at least one monoacylphosphine oxide of formula (I)

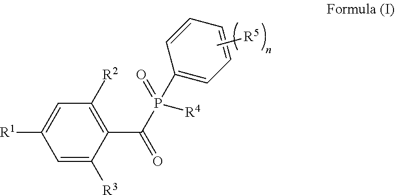

in which
R¹, R², R³ independently of one another are each H, or a linear or branched C₁-C₁₀ alkyl radical or OR⁶,
R⁴ is a linear or branched C₁-C₁₀ alkyl radical, C₁-C₁₀ alkoxy radical, cyclohexyl radical or a phenyl, methylphenyl or mesityl radical,
R⁵ is a linear or branched C₁-C₁₀ alkyl radical,
R⁶ is a linear or branched C₁-C₆ alkyl radical, and
n is 0, 1, 2 or 3,
   and at least one phenolic inhibitor, wherein the molar ratio of inhibitor to photoinitiator is in a range from 0.002 to 0.9. The initiator system is particularly suitable for the photopolymerization of radically polymerizable compositions containing urethane group-containing monomers and for the production of dental moldings with low discoloration.

### CROSS-REFERENCE TO RELATED APPLICATIONS

This application claims priority to European Patent Application No. 23182872.4 filed on Jun. 30, 2023, the disclosure of which is incorporated herein by reference in its entirety.

### TECHNICAL FIELD

The present invention relates to radically polymerizable compositions which are particularly suitable as dental materials for the manufacture of dental moldings, such as artificial teeth, dentures, inlays, onlays, splints, crowns, bridges, veneering materials and orthodontic devices, such as plastic corrective splints, so-called aligners and positioners.

### BACKGROUND

Classical dental polymerization systems usually consist of a mixture of monomers, initiator components, stabilizers and pigments (J. Viohl, K. Dermann, D. Quast, S. Venz, Die Chemie zahnärztlicher Füllungskunststoffe [The chemistry of dental restoratives], Carl Hanser Verlag [Carl Hanser Publishers], Munich-Vienna1986, 21-27). In this context, mixtures of dimethacrylates are mostly used as monomers for the construction of polymer networks (cf. A. Peutzfeldt, Resin composites in dentistry: The monomer systems, Eur. J. Oral. Sci. 105 (1997) 97-116; N. Moszner, T. Hirt, New Polymer-Chemical Developments in Clinical Dental Polymer Materials: Enamel-Dentin Adhesives and Restorative Composites, J. Polym. Sci. Part A: Polym. Chem. 50 (2012) 4369-4402). Examples include the highly viscosity dimethacrylates 2,2-bis[4-(2-hydroxy-3-methacryloyloxypropyl)phenyl]propane (Bis-GMA) and 1,6-bis[2-methacryloyloxyethoxycarbonylamino]-2,4,4-trimethylhexane (UDMA) and the low-viscosity dimethacrylates bismethacryloyloxymethyltricyclo[5.2.1.] decane (TCDMA), decanediol-1,10-dimethacrylate (D₃MA) and triethylene glycol dimethacrylate (TEGDMA). In contrast, the monofunctional monomer methyl methacrylate (MMA), which has a low viscosity but is very volatile, is mainly used for dental prostheses.

Depending on the field of application, further additives can be added. To trigger radical polymerizations by light, photoinitiators for the UVA range or visible range are used, which form radicals when irradiated with UVA light, usually with a wavelength of 365 nm (Hg vapor lamp) to 395 nm (LED), or blue light (400-480 nm).

Prosthetic materials contain powdered polymethyl methacrylate (PMMA) as an essential component, which forms a paste with MMA. Curing takes place thermally or with redox initiators.

A major problem in the radical polymerization of methacrylates is the polymerization shrinkage (ΔV_{P}), i.e. the volume contraction of the methacrylate monomers used during polymerization, which can lead, for example, to very disadvantageous marginal gap formation in filling composites or adversely affects the dimensional stability of denture materials. The polymerization shrinkage ΔV_{P} is e.g. 21.0% by volume for pure MMA.

Another disadvantage of PMMA or dimethacrylate polymers is the great brittleness of the materials. The low fracture toughness is an inherent property of amorphous PMMA glass and, in the case of polymer networks formed by dimethacrylate blends, is mainly caused by their irregular network structure. In addition, methacrylates exhibit incomplete double bond conversion, which in the case of monomethacrylates may result in a significant residual monomer content and thus adversely affect the biocompatibility of the polymers.

Additive manufacturing (AM) processes are increasingly being used to produce dental molded bodies. These are also referred to as generative manufacturing processes or 3D printing and are summarized under the term “rapid prototyping” (RP). Here, three-dimensional models or components are continuously produced layer by layer on the basis of computer-generated design data (CAD data). Common processes are stereolithography (SL), selective laser sintering (SLS), 3D printing, fused deposition modelling (FDM), ink jet printing (IJP), 3D plotting, multi-jet modelling (MJM), solid freeform fabrication (SFF), laminated object manufacturing (LOM) and laser powder forming (LPF). These processes can be used to produce models, structural or molded parts cost-effectively, even in small series (A. Gebhardt, Generative Fertigungsverfahren [Generative manufacturing processes], 3^{rd} ed., Carl Hanser Verlag [Carl Hanser Publishers], Munich 2007, 77ff.).

In stereolithography, a molded part is built up layer by layer from a liquid and curable monomer resin on the basis of CAD data, whereby the curing of the layers takes place by controlled light exposure and thus by photopolymerization (cf. I. Gibson, D. W. Rosen, B. Stucker et al. Gibson, D. W. Rosen, B. Stucker et al, “Additive Manufacturing Technologies”, Volume 238, Springer Verlag [Springer Publishers] (2010)). A UV laser is often used as a light source for this purpose. The DLP (Digital Light Processing) process uses a projectable image to cure the materials layer by layer.

In view of the special characteristics of this process, construction materials for the generative production of dental molded parts must meet a number of different requirements. They should have low viscosity and high transparency and have good mechanical properties after curing. The manufactured molded articles must have good flexural strength and a high modulus of elasticity and, in particular, good fracture toughness.

So-called impact modifiers can be added to polymerization resins to improve fracture toughness. These are usually polymer particles with a core-shell structure.

EP 3 020 361 A1 and corresponding U.S. Pat. No. 9,795,541 B2, which US patent is hereby incorporated by reference, disclose radically polymerizable compositions which are said to be suitable for the production of dental products by generative manufacturing processes and which contain polysiloxanes, disiloxanes and optionally further monomers substituted with radically polymerizable groups, for example urethane (meth)acrylate esters. They are said to have low shrinkage and high dimensional accuracy.

US 2018/0000570 A1, which is hereby incorporated by reference, relates to construction materials based on mono- and multifunctional (meth)acrylates for the generative production of dental components. The construction materials contain silicone-acrylic-based rubber particles with a core-shell structure (product S2006, Mitsubishi Rayon Co.) as impact modifiers and oligomers produced by reacting trimethyl-1,6-diisocyanate, bisphenol A propoxylate and 2-hydroxyethyl methacrylate (HEMA). The cured components are said to have good mechanical and physical properties as well as good biocompatibility.

U.S. Pat. No. 10,299,896 B2 and US 2019/0053883 A1, which are both hereby incorporated by reference, disclose dental components produced by generative processes comprising at least two layers of differently composed construction materials. One layer is formed by a material comprising oligomers obtained by reacting intermediates having terminal isocyanate groups with hydroxyl-based methacrylates, a polymerizable acrylic compound and an impact modifier. At least one further layer is formed by a material containing a urethane monomer, a glycol dimethacrylate and filler. The combination of materials with different mechanical and physical properties is said to be advantageous for adapting the components to different requirements. Commercially available polymers with a core-shell structure are used as impact modifiers, such as the product M570 from Kaneka.

EP 3 564 282 A1 and corresponding U.S. Ser. No. 11/542,362B2, which US patent is hereby incorporated by reference, disclose curable compositions for high temperature photopolymerization processes containing an oligomeric urethane dimethacrylate as a glass transition temperature modifier, a (poly)carbonate (poly)urethane dimethacrylate as a toughness modifier and optionally core-shell particles. They are said to have good thermomechanical properties and biocompatibility and to be suitable for the fabrication of orthodontic appliances.

A major disadvantage for dental applications is that core-shell polymers (CSP) significantly reduce the transparency of the materials, which is detrimental to the stereolithographic building process. A reduction in transparency reduces the depth of cure and can thus lead to a deterioration of the mechanical properties and an increase in the residual monomer content.

From EP 4 085 893 A1 and corresponding US11998621 B2, which US patent is hereby incorporated by reference, radically polymerizable materials for the production of dental moldings by generative processes are known, which contain an ABA or AB block copolymer to improve fracture toughness and fracture work. The materials preferably further comprise 30 to 70% by weight of at least one monofunctional, free-radically polymerizable monomer and at least one free-radically polymerizable urethane di(meth)acrylate telechel having a number average molecular weight of 750 to 2000 g/mol. The materials may contain core-shell polymers to improve fracture toughness and impact strength. They are characterized by a relatively high content of monofunctional monomers.

Monomers containing urethane groups are characterized by very good photoreactivity and produce photopolymers with very good mechanical properties even after storage in water. A disadvantage is that they can lead to severe discoloration of the moldings made from them. The discoloration occurs both during curing of the resins and during storage of the moldings.

The photoinitiators used for curing can also cause discoloration. In particular, camphorquinone and camphorquinone-amine photoinitiator systems, which are frequently used for dental applications, regularly lead to clearly visible money discoloration.

US 2008/0009557 A1, which is hereby incorporated by reference, suggests the addition of iodonium salts, such as [4-(2-hydroxy-1-tetradecyloxy)phenyl]phenyliodonium hexafluoroantimonate, to prevent camphorquinone-related discoloration. The iodonium salts are intended to reduce discoloration by oxidation of the yellow by-products formed during polymerization.

T. Sumiyoshi, W. Schnabel, W.; Henne, A., “Photolysis of acylphosphine oxides II: The influence of methyl substitution in benzoyldiphenylphosphine oxides”, J. Photochem. 32 (1986) 119, investigate the influence of the substitution of hydrogen atoms of the benzoyl group of benzoyldiphenylphosphine oxides by methyl groups on the light-induced α-cleavage of benzoyldiphenylphosphine oxides. It was found that the substitution of the para hydrogen accelerated the cleavage most markedly, while the trisubstituted 2,4,6-trimethyl benzoyldiphenylphosphine oxide showed about the same cleavage rate as the unsubstituted benzoyldiphenylphosphine oxide. Substitution in the ortho position slowed down the cleavage.

C. Dietlin, T. T. Trinh, S. Schweizer, B. Graff, F. Morlet-Savary, P.-A. Noirot, J. Lalevée, “Rational design of acyldiphenylphosphine oxides as photoinitiators of radical polymerization”, Macromolec. 52 (2019) 7886), compare the properties of monoacylphosphine oxides with different substituents on the benzoyl group with those predicted from theoretical calculations. Good agreement was found between measured and calculated values. The photoinitiator reactivity and the double bond conversion correlate strongly with the solubility of the monoacylphosphine oxides.

H. Duan, K. Leng, X. Xu, Q. Li, D. Liu, Y. Han, J. Gao, Q. Yu, Z. Wang, “Monoacylphosphine oxides with substituents in the phosphonyl moiety as Norrish I photoinitiators: Synthesis, photoinitiation properties and mechanism”, J. Photochem. Photobiol. A: Chem. 421 (2021) 113517, investigate the effect of substituents on the phosphonyl group of monoacylphosphine oxides on stability and initiator properties. It was found that both stability and light absorption can be improved by the introduction of methyl groups.

Compared to camphorquinone, monoacylphosphine oxides and bisacylphosphine oxides have only a low intrinsic color, but also frequently lead to yellow discoloration, especially in combination with urethane (meth)acrylates. These are caused by cleavage products of the photoinitiators and their secondary products, such as combination products. Although these colored impurities are degraded over time when the moldings are stored under humid conditions in daylight, this process usually requires periods of several weeks to several months. For practical use as dental materials, however, materials are desired that decolorize after a few days to such an extent that discoloration is no longer visible to the naked eye.

### SUMMARY

It is an object of the invention to provide polymerizable materials for the production of dental moldings, which are color-stable and do not discolor during polymerization or at least decolorize rapidly afterwards. The materials should be suitable for processing with generative methods and, after curing, have good mechanical properties, in particular a high fracture toughness, good flexural strength and a relatively high flexural modulus, be well biocompatible and cure quickly.

### BRIEF DESCRIPTION OF THE DRAWINGS

Exemplary embodiments of the invention are shown in the drawings and are described in more detail below, in which:

FIG. 1 shows the change over time of the b* value according to the CIE-Lab color system of radically polymerizable compositions as a function of the storage time in water at 50° C.

FIG. 2 shows the change over time of the b* value of compositions containing 1.0 wt. % of the photoinitiator TPO and different concentrations of the inhibitor pyrogallol (curves from top to bottom 100, 200, 300, 500 ppm).

FIG. 3 illustrates the determination of the kinetic decolorization vector using the composition with 300 ppm of the inhibitor MEHQ from FIG. 1.

### DETAILED DESCRIPTION

According to the invention, this object is achieved by an initiator system for radical polymerization which comprises at least one monoacylphosphine oxide of the formula (I)

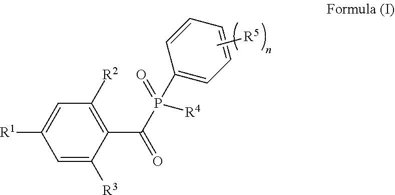

in which
R¹, R², R³ independently of on another are each H, or a linear or branched C₁-C₁₀ alkyl radical or OR⁶,
R⁴ is a linear or branched C₁-C₁₀ alkyl radical, C₁-C₁₀ alkoxy radical, cyclohexyl radical or a phenyl, methylphenyl or mesityl radical,
R⁵ is a linear or branched C₁-C₁₀ alkyl radical,
R⁶ is a linear or branched C₁-C₆ alkyl radical, and
n is 0, 1, 2 or 3, and at least one phenolic inhibitor. The initiator system is characterized in that the molar ratio of inhibitor to photoinitiator is in a range of 0.002 to 0.9, preferably 0.02 to 0.8, particularly preferably 0.05 to 0.2.

Preferred photoinitiators are those of the formula (I) in which the variables have the following meanings:
R¹, R², R³ independently of one another are each a linear or branched C₁-C₃ alkyl radical or OR⁶, preferably methyl or methoxy,
R⁴ is a linear or branched C₁-C₅ alkyl, C₁-C₅ alkoxy, cyclohexyl or phenyl radical, preferably ethoxy, cyclohexyl or phenyl,
R⁵ is a linear or branched C₁-C₃ alkyl radical, preferably methyl,
R⁶ is a linear or branched C₁-C₃ alkyl radical, preferably ethyl, and
n is 0, 1 or 3, preferably 0.

According to a further preferred embodiment, the variables of formula (I) have the following meanings:
R¹, R², R³ independently of one another are each H, a linear or branched C₁-C₃ alkyl radical or OR⁶, preferably H, methyl or methoxy, and particularly preferably two of the radicals R¹, R² and R³ are methoxy and one radical is H or methyl,
R⁴ is a linear or branched C₁-C₅ alkyl, C₁-C₅ alkoxy, cyclohexyl or phenyl radical, preferably tert-butyl, ethoxy, cyclohexyl or phenyl,
R⁵ is a linear or branched C₁-C₃ alkyl radical, preferably methyl,
R⁶ is a linear or branched C₁-C₃ alkyl radical, preferably methyl, and
n is 0, 1, 2 or 3, most preferably 3,
   wherein if R⁴ is not a phenyl radical, n is preferably 0, and if R⁴ is a phenyl radical, n is preferably 1, 2 or 3.

Further preferred are compounds of the formula (I) in which the variables have the following meanings:
R¹ is H, a linear or branched C₁-C₃ alkyl radical or OR⁶, preferably H, methyl or methoxy,
R², R³ independently of one another are each H, a linear or branched C₁-C₃ alkyl radical or OR⁶, preferably H, methyl or methoxy, particularly preferably methyl or methoxy and most preferably methoxy,
R⁴ is a linear or branched C₁-C₅ alkyl, C₁-C₅ alkoxy, cyclohexyl or phenyl radical, preferably ethoxy, cyclohexyl or phenyl,
R⁵ is a linear or branched C₁-C₃ alkyl radical, preferably methyl,
R⁶ is a linear or branched C₁-C₃ alkyl radical, preferably ethyl, and
n is 0, 1 or 3, preferably 0.

Particularly preferably, at least one of R¹, R² and R³ is OR⁶, and according to an even more preferred embodiment, 2 or 3 of R¹ to R³ are OR⁶. Preferably, at least R¹ is OR⁶. Such photoinitiators are characterized by particularly good color stability.

It was surprisingly found that photoinitiators according to formula (1) in combination with a defined amount of selected inhibitors enable the preparation of radically polymerizable compositions which are storage-stable and do not exhibit any discoloration after polymerization without the inhibitors impairing the polymerization. It is particularly advantageous that compositions with radically polymerizable monomers containing urethane groups can also be cured without discoloration.

Radically polymerizable compositions which, in addition to the initiator system according to the invention, contain at least one radically polymerizable monomer containing urethane groups are also an object of the invention.

Particularly preferred are compositions comprising
(a) 0.1 to 8% by weight of at least one monoacylphosphine oxide of formula (1),
(b) 1 to 99% by weight of at least one urethane group-containing monomer, preferably urethane di(meth)acrylate,
(c) 0 to 70% by weight of one or more aliphatic, cycloaliphatic, aromatic, bicyclic or tricyclic mono(meth)acrylates,
(d) 0 to 50% by weight of one or more di(meth)acrylates without urethane groups,
(e) 0 to 12% by weight of one or more block copolymers and/or core-shell polymers,
(f) 200 to 1200 ppm, preferably 300 to 1200 ppm, of at least one phenolic inhibitor,
(g) 0 to 50% by weight of inorganic or organic fillers,
(h) 0 to 30% by weight of other additives.

All weight percentages herein refer in each case to the total mass of the composition, unless otherwise stated.

The amount of monoacylphosphine oxides of formula (I) is preferably chosen to be in the range of 0.1 to 6.0 mol % based on the double bond content of the radically polymerizable components.

Particularly preferred are compositions comprising
(a) 0.30 to 6.5% by weight, preferably 0.5 to 4.0% by weight and more preferably 0.9 to 2.6% by weight of at least one monoacylphosphine oxide of the formula (1),
(b) 1 to 80% by weight, preferably 10 to 75% by weight, of at least one urethane group-containing monomer, preferably urethane di(meth)acrylate,
(c) 0 to 66% by weight, preferably 0 to 60% by weight, more preferably 0 to 5% by weight or 5 to 60% by weight, preferably 40 to 60% by weight, of one or more aliphatic, cycloaliphatic, aromatic, bicyclic or tricyclic mono(meth)acrylates,
(d) 0 to 40% by weight, preferably 0 to 25% by weight, of one or more di(meth)acrylates without urethane groups,
(e) 0 to 10% by weight, preferably 0 to 8% by weight, more preferably 0 to 2% by weight or 4 to 8% by weight of one or more block copolymers and/or core-shell polymers,
(f) 300 to 1000 ppm, preferably 300 to 500 ppm, of at least one phenolic inhibitor,
(g) 0 to 40% by weight, preferably 0 to 25% by weight, of inorganic and/or organic fillers,
(h) 0 to 25% by weight, preferably 0.01 to 10% by weight, of one or more further additives selected from UV absorbers, optical brighteners, colorants, plasticizers and thixotropic additives. The amount specified for the additives refers to the total mass of additives.

In the case of the particularly preferred embodiment, the amount of monoacylphosphine oxides of the formula (1) is preferably chosen to be in the range from 0.3 to 5.0 mol %, more preferably 0.4 to 3.0 mol % and most preferably 0.7 to 2.0 mol %, based on the double bond content of the free-radically polymerizable constituents.

Monoacylphosphine oxides of formula (I) preferred according to the invention are:

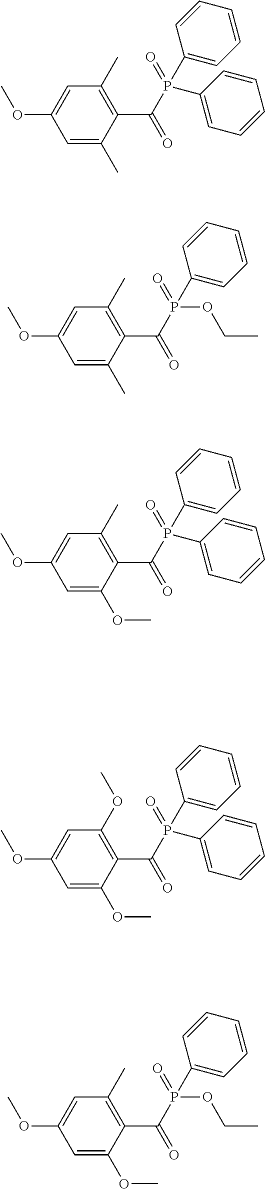

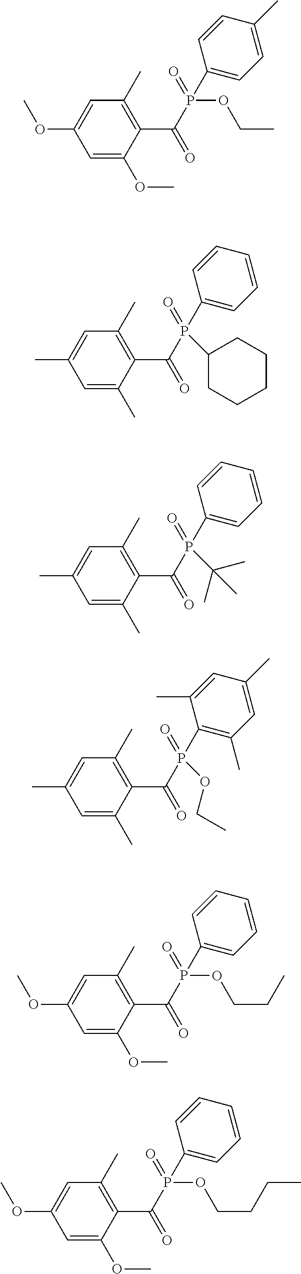

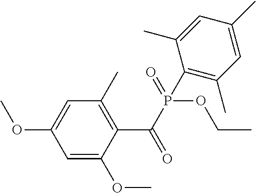

Monoacylphosphine oxides of formula (I) can be prepared by the known methods of organic chemistry, for example by a one-pot synthesis in which in a fist step an appropriately substituted benzaldehyde is reacted in the presence of a base, e.g. triethylamine or sodium carbonate, e.g. in tetrahydrofuran (THF) as solvent with a corresponding phosphine oxide at room temperature or elevated temperature within hours or several days:

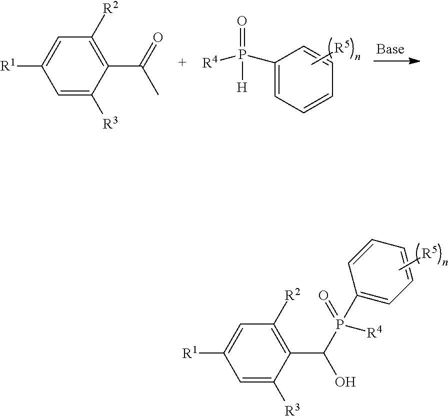

In a second step, after removing the solvent in the absence of light, manganese(IV) oxide in dichloromethane (DOM) is added as an oxidizing agent and then the corresponding monoacylphosphine oxide of formula (1) is obtained after hours or days:

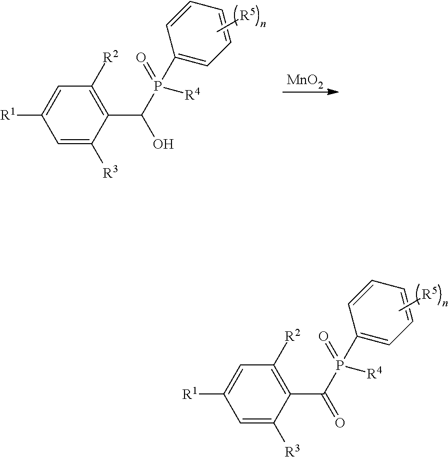

Concrete example:

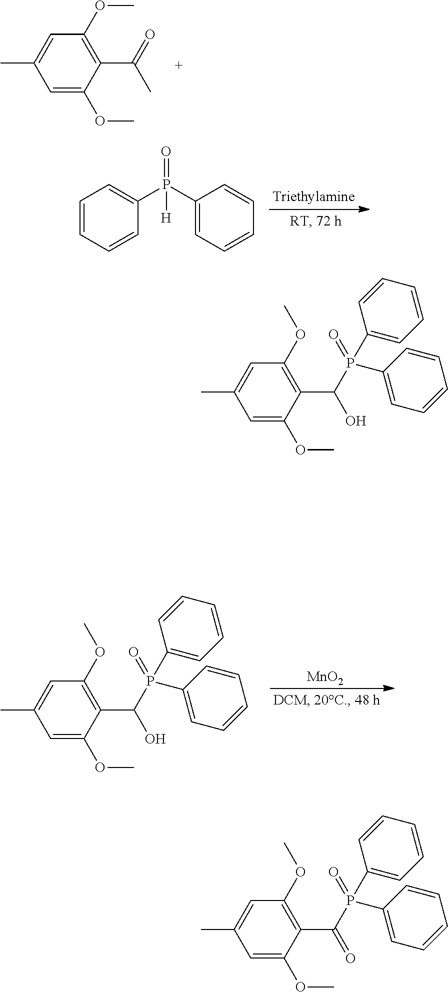

The (meth)acrylate monomer (b) containing urethane groups is preferably a monomer or a mixture of monomers which has/have a urethane group content of 0.01 to 5 mmol/g, preferably 0.1 to 4.5 mmol/g and more preferably 0.5 to 4.0 mmol/g. Particularly preferred are urethane di(meth)acrylates, even more preferred are urethane di(meth)acrylates with a molar mass of less than 4000 g/mol and most preferred are urethane di(meth)acrylates with a molar mass of less than 4000 g/mol, which contain 1 to 8, preferably 1 to 6 and particularly preferably 1 to 4 urethane groups.

(Meth)acrylate monomers containing urethane groups preferred according to the invention are obtainable by:
A) Reaction of 2 moles of 2-isocyanatoethyl (meth)acrylate (R⁸: H or CH₃) with one mole of α,ω-alkanediols, cycloaliphatic, tricyclic or aromatic diols as well as ethoxylated or propoxylated aromatic diols (R⁷: C₁-C₂₀ aliphatic, cycloaliphatic or tricyclic alkylene or C₆-C₁₄-arylene or alkoxylated C₆-C₁₄-arylene):

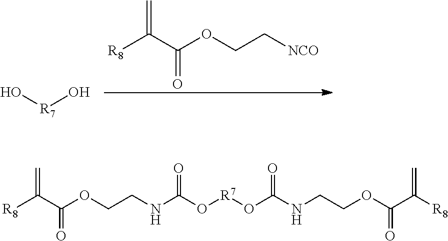

B) Reaction of 1 mol of one or more diisocyanates (R⁹: C₁-C₁₂ aliphatic, cycloaliphatic alkylene or C₆-C₁₄ arylene, which may carry alkyl substituents) with 2 mol of 2-hydroxyalkyl (meth)acrylate, poly(ethylene glycol) or poly(propylene glycol) mono(meth)acrylate and mixtures thereof (R¹⁰: H or CHs; R¹¹: linear or branched C₁-C₄₀ alkylene, wherein the alkylene chain may be interrupted by one or more O atoms):

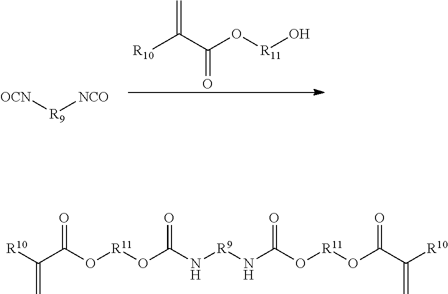

C) Reaction of 1 mol of ethoxylated or propoxylated bisphenol-A, decanediol or dodecanediol or other α,ω-alkanediols, cycloaliphatic, tricyclic or aromatic diols or ethoxy- or propoxylated aromatic diols (R⁷: C₁-C₂₀ aliphatic, cycloaliphatic or tricyclic alkylene or C₆-C₁₄-arylene or alkoxylated C₆-C₁₄-arylene) with 2 moles of isophorone diisocyanate (IPDI) and subsequent reaction with 2 moles of 2-hydroxyethyl (meth)acrylate or hydroxypropyl (meth)acrylate (R¹⁰: H or CH₃; R⁷: linear or branched C₁-C₄₀ alkylene, wherein the alkylene chain may be interrupted by one or more O atoms):

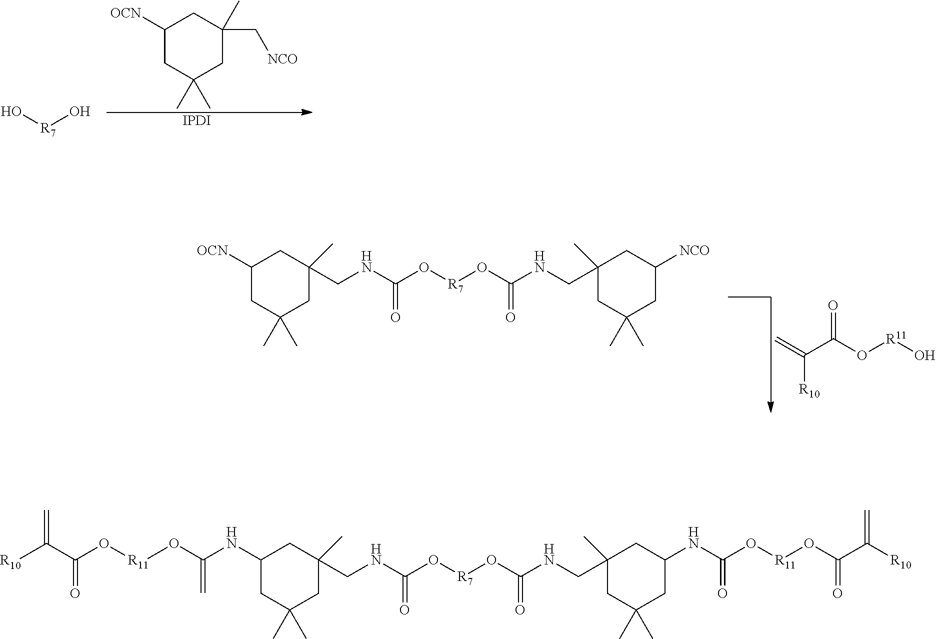

D) Reaction of 1 mole of 2-isocyanatoethyl (meth)acrylate with 1 mole of 2-hydroxyalkyl (meth)acrylate, poly(ethylene glycol) or poly(propylene glycol) mono(meth)acrylate (R¹¹: linear or branched C₁-C₄₀ alkylene, wherein the alkylene chain may be interrupted by one or more O atoms; R⁸, R¹⁰: H or CH₃):

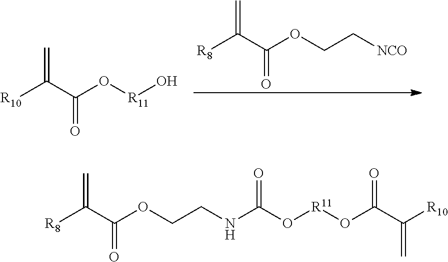

Particularly preferred according to the invention are urethane di(meth)acrylates with a molar mass of less than 750 g/mol, which are obtainable according to synthesis path A by:
Reaction of 2 moles of 2-isocyanatoethyl (meth)acrylate with one mole of α,ω-alkanediols (ethylene glycol, propylene glycol, butane-1,4-diol, pentane-1,5-diol, neopentyl glycol, hexane-1,6-diol, 2,2,4-timethylhexanediol, 2,2,4-trimethyl-1,3-pentanediol, heptane-1,7-diol, octane-1,8-diol, nonaned-1,9-diol, decane-1,10-diol, undecane-1,11-diol or dodecane-1,12-diol), C₆-C₁₈-alkanediols, which may contain 1 to 4 O or S atoms in the carbon chain, cycloaliphatic diols (2-methyl-1,4-cyclopentanediol, 1,3- or 1,4-cyclohexanediol, cyclooctanediol, methylcycloheptanediol, 2,5-dimethyl-1,4-cyclohexanediol, 3,3,5-trimethylcyclopentane-1,2-diol, 2,2,4,4-tetramethyl-1,3-cyclobutanediol, tricyclic (tricyclodecanedimethanol: tricyclo[5.2.1.0^{2,6}]decanedimethanol) or aromatic ethoxy- or propoxylated diols with on average 2, 3 or 4 ethoxy or propoxy groups (bisphenol-A, bisphenol-B, bisphenol-E, bisphenol-F, bisphenol-S, hydroquinone, resorcinol, 2,6-dihydroxynaphthalene or 4,4′-dihydroxybiphenyl).

Also particularly preferred are urethane di(meth)acrylates with a molar mass of less than 750 g/mol, which are obtainable according to synthesis path B by: Reaction of 1 mol of hexamethylene-1,6-diisocyanate (HMDI), 2,2,4-trimethylhexamethylene-1,6-diisocyanate (TMDI), isophorone diisocyanate (IPDA), diphenylmethane-4,4′-diisocyanate (4,4′-MDI), hydrogenated diphenylmethane-4,4′-diisocyanate (H12-MDI: 4,4′-methylene bis(cyclohexyl isocyanate) or tetramethylxylylene diisocyanate (TMXDI: 1,3-bis(2-isocyanatopropan-2-yl) benzene) or mixtures thereof with 2 mol of 2-hydroxyethyl (meth)acrylate, hydroxypropyl (meth)acrylate or poly(ethylene glycol)-200- or poly(propylene glycol)-200-mono(meth)acrylate.

As urethane group-containing (meth)acrylate monomers (b), urethane di(meth)acrylates with a molar mass of less than 1000 g/mol are further preferred, which are obtainable, for example, by reacting a diisocyanate with 2 mol of a hydroxyl group-containing (meth)acrylate or by reacting a diol with 2 mol of an isocyanate group-containing (meth)acrylate.

Particularly preferred are urethane di(meth)acrylates with a molar mass of less than 1000 g/mol, which are obtainable by reacting 1 mol of hexamethylene-1,6-diisocyanate (HMDI), 2,2,4-trimethylhexamethylene-1,6-diisocyanate (TMDI), isophorone diisocyanate (IPDA), diphenylmethane-4,4′-diisocyanate (4,4′-MDI), hydrogenated diphenylmethane-4,4′-diisocyanate (H12-MDI: 4,4′-methylene bis(cyclohexyl isocyanate) or tetramethylxylylene diisocyanate (TMXDI: 1,3-bis(2-isocyanatopropan-2-yl)benzene), or a mixture thereof, with 2 moles of 2-hydroxyethyl(meth)acrylate, hydroxypropyl(meth)acrylate, poly(ethylene glycol)-200- or poly(propylene glycol)-200-mono(meth)acrylate.

Also particularly preferred are urethane di(meth)acrylates with a molar mass of less than 1000 g/mol which are obtainable by reacting 2 moles of 2-isocyanatoethyl (meth)acrylate with one mole of a α,ω-alkanediol, preferably ethylene glycol, propylene glycol, butane-1,4-diol, pentane-1,5-diol, neopentyl glycol, hexane-1,6-diol, 2,2,4-trimethylhexanediol, 2,2,4-trimethyl-i1,3-pentanediol, heptane-1,7-diol, octane-1,8-diol, nonane-1,9-diol, decane-1,10-diol, undecane-1,11-diol or dodecane-1,12-diol, a C₆-C₁₈-alkanediol containing 1 to 4 O or S atoms in the carbon chain, a cycloaliphatic diol, preferably 2-methyl-i1,4-cyclopentanediol, 1,3- or 1,4-cyclohexanediol, cyclooctanediol, methylcycloheptanediol, 2,5-dimethyl-1,4-cyclohexanediol, 3,3,5-trimethylcyclopentane-1,2-diol or 2,2,4,4-tetramethyl-1,3-cyclobutanediol, of a tricyclic diol, preferably tricyclodecanedimethanol or tricyclo[5.2.1.0^{2,6}]decanedimethanol, of an aromatic ethoxy- or propoxylated diol having on average 2, 3 or 4 ethoxy or propoxy groups, preferably bisphenol-A, bisphenol-B, bisphenol-E, bisphenol-F, bisphenol-S, hydroquinone, resorcinol, 2,6-dihydroxynaphthalene, 4,4′-dihydroxybiphenyl, or a mixture thereof.

Other preferred urethane di(meth)acrylates are the urethane di(meth)acrylate telechels disclosed in EP 4 085 893 A1. These are obtainable by reacting diisocyanates with diols (HO-DA-OH) and then reacting the α,ω-isocyanate-functionalized urethane telechels with 2-hydroxyethyl methacrylate (HEMA) or hydroxypropyl methacrylate (HPMA). DA stands for an aromatic or aliphatic hydrocarbon radical having 6 to 33 carbon atoms, preferably a divalent polycyclic hydrocarbon radical, in particular an o-diphenyl, p-diphenyl or bisphenol A radical, or a branched or preferably linear C₂-C₁₈ alkylene group. The hydrocarbon radicals may contain one or more O atoms and/or S atoms, with O atoms being preferred.

Telechels with a molecular weight of 800 to 4000 g/mol are preferred, particularly preferably with a molecular weight of 1000 to 3800 g/mol and most preferably with a molecular weight of 1000 to 2000 g/mol. Further preferred are telechelic compounds containing 1 to 8, especially preferably 4 to 6, urethane groups.

Preferred diols of the formula HO-DA-OH are ethoxylated or propoxylated bisphenol-A, o-diphenyl or p-diphenyl with 2 to 6 ethoxy or propoxy groups as well as C₂-C₁₈-alkanediols which may contain 1 to 4 O or S atoms in the carbon chain. Particularly preferred diols are ethoxylated or propoxylated bisphenol-A with 2, 3 or 4 ethoxy or propoxy groups, hexane-1,6-diol, octane-1,8-diol, nonane-1,9-diol, decane-1,10-diol or dodecane-1,12-diol, tetra- or pentaethylene glycol. Ethoxylated or propoxylated bisphenol-A with 2 or 3 ethoxy or propoxy groups, decane-, undecane- or dodecanediol as well as cyclic or polycyclic aliphatic diols, in particular cyclohexanediol, norbornanediol, tricyclodecanediol and tricyclodecanedimethanol (octahydro-4,7-methano-1H-indenedimethanol) are particularly preferred.

Preferred diisocyanates are hexamethylene-1,6-diisocyanate (HMDI), 2,2,4-trimethylhexamethylene-1,6-diisocyanate (TMDI), 1-isocyanato-3-isocyanatomethyl-3,5,5-trimethylcyclohexane (isophorone diisocyanate, IPDI), m-tetramethylxylylene diisocyanate, (1,3-bis(2-isocyanato-2-propyl)benzene, TMXDI), toluene-2,4-diisocyanate (TDI), diphenylmethane-4,4′-diisocyanate (MDI) and 1-isocyanato-4-[(4-isocyanatocyclohexyl)methyl]cyclohexane (H12MDI), IPDI being particularly preferred.

Particularly preferred are telechels according to the general formula (II)

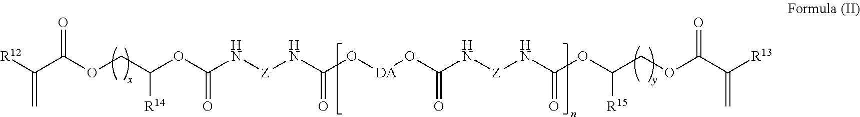

in which the variables have the following meanings:
R¹², R¹³ independently of one another are each H or methyl, preferably methyl,
R¹⁴, R¹⁴ independently of one another are each H or methyl, preferably methyl,
x, y independently of one another are each an integer from 1 to 11, preferably 1 to 5,
n is 1, 2 or 3, preferably 1,
Z is

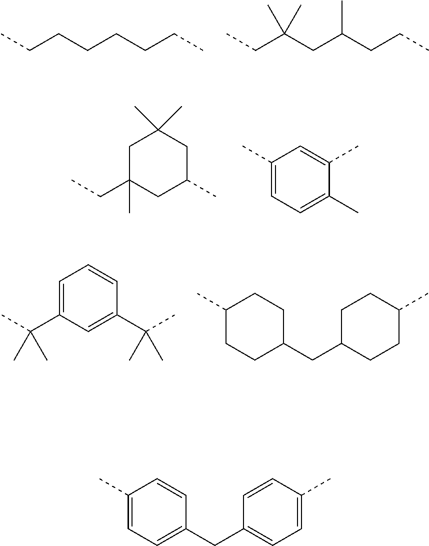

Preferably:

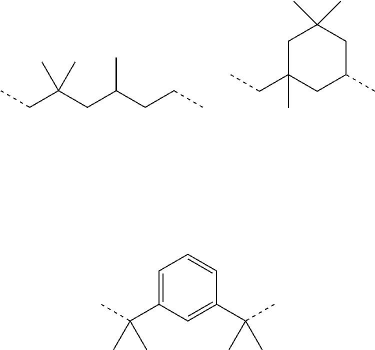

DA is a structural element derived from the diols HO-DA-OH by splitting off the hydrogen atoms of the two hydroxyl groups.

The urethane di(meth)acrylate telechels carry two radically polymerizable (meth)acrylate groups and are suitable as crosslinkers. They are characterized by a good radical polymerization ability and, due to their relatively high molar mass, yield polymers with a lower network density and low polymerization shrinkage.

Telechels with a molar mass of 1000 to 3800 g/mol, which contain a maximum of 6 urethane groups, are particularly preferred. The urethane di(meth)acrylate telechels preferred according to the invention are characterized by a good radical polymerization ability. They also impart good cohesive properties to the cured materials.

Particularly preferred urethane di(meth)acrylates are UDMA (=1,6-bis-[2-methacryloyloxyethoxycarbonylamino]-2,4,4-trimethylhexane=addition product of 1 mol 2,2,4-trimethylhexamethylene-1,6-diisocyanate and 2 mol 2-hydroxyethylmethacrylate), V-380 (addition product of 1 mol α,α,α′,α′-tetramethyl-m-xylylene diisocyanate and a mixture of 1.4 mol 2-hydroxyethylmethacrylate and 0.6 mol 2-hydroxypropylmethacrylate), and V-818 (addition product of 1 mol tricyclo [5.2.1.0^{2,6}]decanedimethanol and 2 mol 2-isocyanatoethylmethacrylate). UDMA has a urethane group content of 4.235 mmol/g.

The monomers (b) containing urethane groups, which are preferred according to the invention, are characterized by a good radical polymerization ability. They also impart good cohesive properties to the cured materials.

Unless otherwise stated, the molar mass of oligomers and polymers herein is the number average molar mass Mₙ, determined by gel permeation chromatography (GPC).

Gel permeation chromatography (GPC) is a relative method in which molecules are separated on the basis of their size, more precisely on the basis of their hydrodynamic volume. The absolute molar mass is determined by calibration with known standards. Preferably, narrowly distributed polystyrene standards are used as calibration standards. These are commercially available. Styrene-divinylbenzene columns are used as separation material and tetrahydrofuran (THF) as eluent. Styrene-divinylbenzene columns are suitable for organic soluble synthetic polymers. The measurement is carried out with diluted solutions (0.05-0.2 wt. %) of the polymers to be investigated.

Alternatively, the number-average molar mass can be determined with the known methods of freezing point depression (cryoscopy), boiling point elevation (ebullioscopy) or from the depression of the vapor pressure (vapor pressure osmometry). These are absolute methods that do not require calibration standards. Concentration series of 4 to 6 diluted polymer solutions with concentrations of 0.005 to 0.10 mol/kg are examined and then the measured values are extrapolated to a concentration of 0 mol/kg.

The compositions according to the invention may contain up to 60% by weight of mono(meth)acrylates (c). The mono(meth)acrylate(s) are preferably selected from aliphatic, cycloaliphatic, aromatic, bicyclic and tricyclic mono(meth)acrylates, the total amount of mono(meth)acrylates not exceeding the above mentioned ranges. Particularly preferred are aliphatic mono(meth)acrylates or mono(meth)acrylates having a cycloaliphatic, aromatic, bicyclic or tricyclic group, such as benzyl, tetrahydrofurfuryl or isobornyl (meth)acrylate, 2-[(methoxycarbonyl)amino]ethyl methacrylate, 2-[(propoxycarbonyl)amino]ethyl methacrylate, 2-[(isopropoxycarbonyl)amino]ethyl methacrylate, 2-[(butoxycarbonyl)amino]ethyl methacrylate, 2-[(hexyloxycarbonyl)amino]ethyl methacrylate, 2-[(cyclohexyloxycarbonyl)amino]ethyl methacrylate, 2-[(ethylcarbamoyl)oxy]ethyl methacrylate, 2-[(propylcarbamoyl)oxy]ethyl methacrylate, 2-[(isopropylcarbamoyl)oxy]ethyl methacrylate, 2-[(hexylcarbamoyl)oxy]ethyl methacrylate, 2-[(2-tetrahydrofurfuryloxycarbonyl)amino]ethyl methacrylate, 2-(2-oxo-[1,3]-dioxolan-4-ylmethoxycarbonylamino)ethyl methacrylate, methacrylic acid 2-(furan-2-yl-methoxycarbonylamino)ethyl ester, (1,3-dioxolanon-2-on-4-yl)methyl methacrylate, 2-phenoxyethyl (meth)acrylate, 2-(o-biphenyloxy)ethyl (meth)acrylate, 2-hydroxy-3-phenoxypropyl (meth)acrylate, 2-[(benzyloxycarbonyl)amino]ethyl (meth)acrylate, 2-[(benzylcarbamoyl)oxy]ethyl (meth)acrylate, 1-phenoxypropan-2-yl (meth)acrylate and 2-(p-cumyl phenoxy)ethyl (meth)acrylate, tricylodecane (meth)acrylate, tricyclodecanemethyl (meth)acrylate and 4,7,7-trimethylbicyclo[2.2.1]heptanyl (meth)acrylate.

According to the invention, low-volatility mono(meth)acrylates (c) are preferred. Low-volatility substances are compounds with an evaporation number greater than 35. The evaporation number (VD) indicates how quickly a substance evaporates at room temperature. It is determined according to DIN 53170. The time in which a substance evaporates completely (evaporation time=VDZ) is related to the time that diethyl ether takes to evaporate. The higher the evaporation number, the slower a substance evaporates.

Very particularly preferred monofunctional monomers are tetrahydrofurfuryl and isobornyl (meth)acrylate, 2-[(benzyloxycarbonyl)amino]ethyl methacrylate, 2-[(ethylcarbamoyl)oxy]ethyl methacrylate, 2-[(benzylcarbamoyl)oxy]ethyl methacrylate, methacrylic acid 2-(furan-2-yl-methoxycarbonylamino)ethyl ester, 2-phenoxyethyl (meth)acrylate, 2-(o-biphenyloxy)ethyl (meth)acrylate, 2-hydroxy-3-phenoxypropyl (meth)acrylate, 1-phenoxypropan-2-yl (meth)acrylate, 2-(p-cumylphenoxy)ethyl (meth)acrylate, tricylodecane (meth)acrylate, tricyclodecanemethyl (meth)acrylate, 4,7,7-trimethylbicyclo[2.2.1]heptanyl (meth)acrylate and mixtures thereof.

Aliphatic, cycloaliphatic, aromatic, bicyclic and tricyclic mono(meth)acrylates (c) are characterized by good radical polymerization ability. In addition, the polymers of these mono(meth)acrylates have comparatively low polymerization shrinkage and good mechanical properties. Due to their relatively high molar mass (150 to 350 g/mol) and their relatively non-polar structure, the mono(meth)acrylates preferred according to the invention also have a low volatility and a comparatively low viscosity. However, mono(meth)acrylates lead to a reduction in network density. The proportion of mono(meth)acrylates is therefore limited to a maximum of 60% by weight, preferably a maximum of 55% by weight and particularly preferably a maximum of 50% by weight. Very particularly preferred are compositions which contain a maximum of 5% by weight and most preferably no monofunctional monomers.

The compositions according to the invention may contain as component (d) 0 to 50% by weight and preferably 0 to 40% by weight of one or more di(meth)acrylates without urethane groups. Preferred di(meth)acrylates (d) are bisphenol A di(meth)acrylate, bis-GMA (an addition product of (meth)acrylic acid and bisphenol A diglycidyl ether), ethoxy- or propoxylated bisphenol A di(meth)acrylate having an average of 2, 3 or 10 ethoxy or propoxy groups such as a bisphenol A di(meth)acrylate having 3 ethoxy groups, or 2,2-bis[4-(2-(meth)acryloyloxypropoxy)phenyl]propane, di-, tri- or tetraethylene glycol di(meth)acrylate, trimethylolpropane tri(meth)acrylate, pentaerythritol tetra(meth)acrylate and glycerol di- and tri(meth)acrylate, 1,4-butanediol di(meth)acrylate, 1,10-decanediol di(meth)acrylate, bis[(meth)acryloyloxymethyl]tricyclo-[5.2.1.0^{2,6}]decane, polyethylene glycol or polypropylene glycol di(meth)acrylate, such as polyethylene glycol 200- or -400-di(meth)acrylate or 1,12-dodecanediol di(meth)acrylate. Preferred di(meth)acrylates (d) are bis-GMA, ethoxy- or propoxylated bisphenol A di(meth)acrylate with on average 2 or 3 ethoxy or propoxy groups, tri- or tetraethylene glycol di(meth)acrylate, glycerol di- and tri(meth)acrylate, 1,4-butanediol di(meth)acrylate, 1,10-decanediol di(meth)acrylate, bis[(meth)acryloyloxymethyl]tricyclo-[5.2.1.0^{2,6}]decane, 1,12-dodecanediol di(meth)acrylate. Particularly preferred di(meth)acrylates (d) are: Bis-GMA, ethoxy- or propoxylated bisphenol A di(meth)acrylate with on average 2 or 3 ethoxy or propoxy groups, triethylene glycol di(meth)acrylate, 1,10-decanediol di(meth)acrylate, bis[(meth)acryloyloxymethyl]tricyclo-[5.2.1.0^{2,6}]decane, 1,12-dodecanediol di(meth)acrylate.

The preferred di(meth)acrylate monomers (d) are characterized by a relatively low molecular weight, di(meth)acrylates (d) with a molecular weight in the range of 200 to 800 g/mol and in particular 220 to 650 g/mol being particularly preferred. Due to the low molecular weight of the di(meth)acrylate monomers (d), in particular in comparison with the urethane di(meth)acrylate telechels (b), the di(meth)acrylate monomers (d) cause a relatively strong crosslinking of the polymers. The proportion of further di(meth)acrylates (d) is therefore limited to a maximum of 50% by weight, preferably to a maximum of 40% by weight. Furthermore, it is preferred that the compositions according to the invention contain, in addition to the urethane (meth)acrylates (b) and the di(meth)acrylates (d), a maximum of 2% by weight, particularly preferably a maximum of 1% by weight, of further polyfunctional monomers, such as, for example, tri-, tetra- or higher-functional (meth)acrylates. According to a particularly preferred embodiment, the dental materials according to the invention contain exclusively urethane di(meth)acrylates (b) as crosslinking agents.

Urethane di(meth)acrylates (b), monofunctional methacrylates (c), and di(meth) acrylate monomers (d) are preferably used in a ratio such that polymers with a network density of vc=800 to 16,000 mol/m³, preferably 2000 to 10,000 mol/m³, particularly preferably 2500 to 8000 mol/m³ are obtained. According to the invention, the crosslink density is mainly adjusted by the ratio of crosslinking to non-crosslinking monomers.

Preferred dental materials according to the invention are those in which the mole fraction of the crosslinking monomers is in a range from 0.30 to 1.0, particularly preferably 0.5 to 0.95. Very particularly preferred are compositions which contain a maximum of 5% by weight or no monofunctional monomers.

All radically polymerizable components of the materials according to the invention are used to calculate the mole fraction, i.e. in particular components (b), (c) and (d). Cross-linking monomers are understood to be all radically polymerizable components which have two or more radically polymerizable groups, i.e. in particular components (b) and (d). Cross-linking monomers are also referred to as polyfunctional monomers. Monofunctional monomers are monomers with only one radical polymerizable group. The network density corresponds to the number of network nodes (in mol) per unit volume and can be calculated by dynamic-mechanical measurements from the plateau value of the storage modulus G′ in the elastic range. The glass transition temperature Tg and the network density vc are determined with a rheometer, preferably an Anton Paar Rheometer MCR301. For this purpose, storage and loss modulus of a test specimen (25×5×1 mm, clamped longitudinally) are measured between 25° C. and 250° C. (frequency 1 Hz, deformation 0.05%, heating rate 2K/min).Tg is the maximum of the loss factor tan 5 (ratio of loss modulus to storage modulus). The network density is calculated according to the formula vc=G′/(RT), where G′ is the storage modulus at the temperature Tg+50 K, R as the general gas constant and T as the temperature at Tg+50 K in Kelvin.

The compositions according to the invention may contain from 0 to 12% by weight and preferably from 0 to 10% by weight of one or more block copolymers and/or core-shell polymers (e) as impact modifiers.

Block copolymers are macromolecules consisting of two or more covalently bonded homopolymer blocks. Block copolymers preferred according to the invention are AB diblock and in particular ABA triblock copolymers.

The A block is a polymerizate, preferably an oligomer, composed of one or more of the following monomers: cyclic aliphatic esters or ethers, arylene oxide, alkylene oxide, free-radically polymerizable monomers, for example α,β-unsaturated acids and α,β-unsaturated acid esters. Preferably, the block A is a poly(meth)acrylate, polylactone, polyphenylene oxide or polyalkylene oxide oligomer. More preferably, the A block is a polymerizate of caprolactone, 2,6-dialkyl-1,4-phenylene oxide and in particular of 2,6-dimethyl-1,4-phenylene oxide, ethylene oxide, propylene oxide or (meth)acrylates. The A block is thus preferably a polycaprolactone (PCL), poly(2,6-dimethyl-1,4-phenylene oxide), poly(ethylene oxide), poly(propylene oxide) or poly(meth)acrylate oligomer.

The B block is preferably a polysiloxane and/or a polyvinyl and/or a polyalkene and/or a polydiene oligomer or a hydrogenated polydiene oligomer. Particularly preferably, the B block is a polydiene oligomer or a hydrogenated polydiene oligomer, polyvinylalkanoate oligomer or a polysiloxane oligomer according to the formula —O—(SiR¹⁶₂—O)ₚ—, in which
R¹⁶ is a linear C₁-C₂₀ alkyl, branched C₃-C₁₂ alkyl or C₆-C₂₀ aryl group, wherein the individual R¹⁶ radicals may be the same or different, and
p is a number from 3 to 100, preferably a number from 10 to 50.

More preferably, the B-block is a polymerizate of dimethylchlorosilane, cyclotri- or cyclotetradimethoxysilane, isoprene, vinyl acetate, isobutene, cis-butadiene or ethylene. The B block is thus preferably a poly(dimethylsiloxane) (PDMS), hydrogenated poly(isoprene), poly(vinyl acetate), hydrogenated poly(isobutene), hydrogenated cis-poly(butadiene) or poly(ethylene) oligomer.

The B blocks are characterized by a relatively high flexibility. Flexible blocks are understood to be blocks formed from monomers whose homopolymers have a glass transition temperature TG below 50° C., preferably below 0° C. and most preferably in the range of −30 to −110° C. Block copolymers with flexible blocks improve the fracture toughness, but affect the flexural strength and the modulus of elasticity of the polymers significantly less than internal plasticizers.

Preferred are ABA and AB block copolymers in which the A block or blocks are preferably each an oligomeric polycaprolactone, poly(2,6-dimethyl-1,4-phenylene oxide), poly(ethylene oxide), poly(propylene oxide), poly(meth)acrylate or poly(meth)acrylate-styrene copolymer building block, poly(meth)acrylate or poly(meth)acrylate-styrene copolymer building block, and the B block is preferably a flexible poly(dimethylsiloxane), poly(isoprene) or hydrogenated poly(isoprene), poly(vinyl acetate), poly(isobutene), cis-poly(butadiene) or hydrogenated cis-poly(butadiene) or a poly(ethylene) building block.

Particularly preferred according to the invention are ABA block copolymers in which the A blocks are each an oligomeric polycaprolactone, poly(meth)acrylate or poly(meth)acrylate-styrene copolymer building block and the B block is a flexible poly(dimethylsiloxane), hydrogenated poly(isoprene) or hydrogenated cis-poly(butadiene) building block.

Very particularly preferred are polyester-polysiloxane block copolymers according to the following general formula:

(PCL)_{q}-b-(PDMS)ᵣ-b-(PCL)_{q}

in which
q is in each case a number from 5 to 40, preferably 10 to 20, and
r is a number from 10 to 100, preferably 30 to 60.

(PCL)_{q} stands for polycaprolactone, which is composed of q caprolactone monomers, and (PDMS)r stands for poly(dimethylsiloxane), which is composed of r dimethylsiloxane monomers. The letter b stands for block.

Further preferred are poly(meth)acrylate-polysiloxane block copolymers containing a polymethyl methacrylate residue as the A block and a polysiloxane residue as the B block, wherein the polysiloxane residue is preferably as defined above and most preferably a poly(dimethylsiloxane) residue.

Particularly preferred are also the ABA triblock copolymers PCL-b-PDMS-b-PCL and PMMA-b-PDMS-b-PMMA with a molar ratio A: B of 0.1 to 5 and with a molar mass of preferably 3 to 25 kDa, particularly preferably 4 to 20 kDa and most preferably 5 to 10 kDa. A preferred block copolymer is PCL-b-PDMS-b-PCL, wherein the PDMS blocks have a molar mass of about 3200 g/mol and the PCL blocks each have a molar mass of about 1600 g/mol. PCL stands for polycaprolactone, PDMS for poly(dimethylsiloxane) and PMMA for polymethyl methacrylate.

Block copolymers can be prepared by the known methods of living or controlled polymerization, e.g. by free radical or ionic (anionic and cationic) polymerization and also by metal-catalyzed ring-opening polymerization of cyclic monomers, such as caprolactone, with controlled free radical polymerization, living anionic polymerization and ring-opening polymerization being preferred. However, block copolymers can also be obtained by linking end groups of homopolymers. The block copolymers used according to the invention can be di- and tri-block copolymers.

AB block copolymers can be prepared, for example, by linking an A block with a terminal OH group by esterification with a B block that has a COOH group. End-group functionalized homopolymer blocks can be prepared relatively easily by the methods of controlled free radical polymerization or by end-capping in anionic polymerization. For example, monomer A is anionically polymerized and an OH group is introduced by endcapping. The OH end group can then be esterified with, for example, a-bromoisobutyric acid. The resulting bromine end group then acts as the starting center for the formation of the B block by ATRP (Atom Transfer Radical Polymerization) of monomer B, initiated by metal complexes of, for example, Cu(I), Ru(I) or Fe(II).

Triblock copolymers can be produced in an analogous way. For example, a B block is produced by anionic polymerization of monomer B via a dianion mechanism. The B middle block formed carries an anion end group on both sides, which initiates the anionic polymerization of monomer A to form the two A blocks (method 1). The esterification of a telechelic B block, which carries a suitable functional group at each end, e.g. an OH group, with two A blocks functionalized only on one side, e.g. with a COOH group, yields ABA triblock copolymers (method 2). Finally, OH-telechelic homopolymers of monomer B can be esterified with a-bromoisobutyric acid. The two bromine end groups formed in this way in homopolymer block B can then be used as the starting center for the formation of the two A blocks by ATRP (method 3).

During the synthesis of the block copolymers, terminal or lateral (meth)acrylate groups that are capable of polymerization can also be introduced. These cause a better integration of the block copolymers into the formed polymer networks through radical copolymerization of the (meth)acrylate groups.

The monomers are preferably chosen so that the A block is miscible with the resin matrix, i.e. the mixture of components (a) to (d), and the B block is not miscible with the resin matrix. Miscibility is understood here in terms of thermodynamics in relation to single phase. Accordingly, a miscible polymer block is understood to be a polymer block of a monomer whose homopolymer is soluble in the resin matrix such that the blend has a transparency of at least 95%. If, on the other hand, the mixture is cloudy or opaque, i.e. if the transparency is significantly less than 95%, the homopolymer and thus the corresponding polymer block is not miscible with the resin matrix. The transparency is measured with a spectrophotometer on 1 mm thick test specimens polished to a high gloss in transmission (D₆₅) according to the standard ISO 10526:1999, e.g. with a Konika-Minolta spectrophotometer of the type CM-5.

The compositions according to the invention may contain, as fracture toughness modifier, alternatively or preferably in addition to the block copolymers, one or more particulate core-shell polymers. Preferred are polymer particles having a soft core, preferably of polybutadiene, hydrogenated polybutadiene, polyisoprene, hydrogenated polyisoprene, polybutyl acrylate, MMA-butadiene-styrene copolymers (MBS) or polydimethylsiloxane, and a hard shell, preferably of PMMA or an MMA-styrene copolymer.

Soft or flexible polymers are understood to be polymers with a glass transition temperature TG below 50° C., preferably below 0° C. and most preferably in the range of −30 to −110° C. A preferred concrete example is PDMS with a TG of about −110° C. By hard polymers is meant polymers with a glass transition temperature above 50° C. and preferably above 80° C. A preferred concrete example is PMMA with a TG of 105-120° C.

The fracture toughness modifying effect of CSP particles in free-radical di(meth)acrylate polymer networks depends primarily on the type of CSP particles, the particle size, the cross-linking density and the weight ratio of core to shell, which is preferably in a range of 1:1 to 200:1. The cross-linking density is largely determined by the proportion of cross-linking monomers in the particle core. This is preferably in a range from 1 to 10% by weight, based on the mass of the core. According to the invention, particles with a particle size of 0.20 to 50.0 μm are preferred. When incorporating the CSP particles into the dental material, care must be taken to ensure good dispersion.

The optional fracture toughness modifier(s) are preferably used in an amount of 1 to 10% by weight, more preferably in an amount of 2 to 8% by weight and most preferably 3 to 6% by weight, based on the total weight of the dental material, if required.

Core-shell polymers have the disadvantage that they can impair the transparency of the compositions, which can have a negative effect on the depth of cure during photopolymerization and also on the aesthetics in the case of dental moldings. Therefore, according to the invention, materials are preferred which contain a maximum of 3% by weight and particularly preferably no core-shell particles.

The compositions according to the invention contain at least one phenolic inhibitor (f). Inhibitors are understood to be radical scavengers that form new, stable radicals with radicals, which then do not react further. Inhibitors are also called polymerization inhibitors and are used to improve the storage stability of radically polymerizable materials. Surprisingly, it was found that phenolic inhibitors in an amount of 300 to 1200 ppm not only improve the storage stability of free-radically polymerizable compositions, but can also prevent discoloration caused by urethane group-containing monomers and photoinitiators, especially in combination with photoinitiators according to formula (1). Phenolic inhibitors, are phenolic derivatives containing a benzene ring substituted with at least one free OH group.

Preferred inhibitors are hydroquinone monomethyl ether (MEHQ, CAS: 150-76-5), 2,6-di-tert-butyl-4-methyl-phenol (BHT, CAS: 128-37-0), 2,6-di-tert-butylphenol (CAS: 128-39-2), pyrogallol (CAS: 87-66-1), hydroquinone (CAS: 123-31-9), 2-tert-butylhydroquinone (CAS: 1948-33-0), 4-tert-butylcatechol (CAS: 98-29-3) or 6-tert-butyl-2,4-xylenol (CAS: 1879-09-0), MEHQ, pyrogallol and BHT being particularly preferred.

The photoinitiator systems according to the invention enable the production of polymerizates that show no or only a slight yellowing directly after polymerization or a short storage time. Discoloration is determined by a stress test, in which the cured polymer is stored in water at 50° C. The color is determined according to the CIE-Lab color system. CIE stands for International Commission on Illumination. Here, the color in the color space is described by 3 coordinates that are at right angles to each other: Lightness L*, which ranges from 0 (pure black) to 100 (pure white). a* represents the red-green axis, where negative values are green and positive values are red. b* represents the yellow-blue axis, where negative values are blue and positive values are yellow. Color differences, the color distance, can be evaluated with the so-called Euclidean distance Delta E* (AE*), where E stands for perception. For the evaluation of discoloration of dental materials, the yellow coloration and thus the b* value is of particular practical relevance.

The materials according to the invention are characterized by having a b* value <4.5 after polymerization, preferably <4 and particularly preferably <3. This value is achieved directly after polymerization or after only a short storage period, preferably after a maximum of 200 hours. This means that polymerization-related discolorations are largely degraded within 200 h. The time course of the decrease in yellow discoloration can be described by the kinetic decolorization vector KDV, which is determined in the manner described in the examples. The compositions according to the invention preferably have a kinetic decolorization vector of less than 3.5, more preferably less than 3.0 and most preferably less than 2.5.

The initiator systems according to the invention also enable the production of radically polymerizable materials with good photopolymerization reactivity and good mechanical properties after curing. After photopolymerization, the materials preferably have a flexural strength of greater than 40 MPa, preferably 40 MPa to 400 MPa, particularly preferably 60 MPa to 400 MPa, and a flexural modulus of greater than 1.5 GPa, preferably 1.5 GPa to 6 GPa, measured according to ISO 20795-1. The materials are color stable and storage stable and exhibit a high polymerization rate and a high double bond conversion.

The compositions according to the invention may contain one or more fillers (g), preferably particulate or fibrous inorganic or organic fillers or composite fillers. Fillers allow the mechanical properties to be influenced. Particularly preferred are inorganic particulate fillers.

Preferred inorganic fillers are oxides, such as SiO₂, ZrO₂ and TiO₂ or mixed oxides of SiO₂, ZrO₂, ZnO and/or TiO₂, nanoparticulate or microfine fillers, such as fumed silica or precipitated silica, glass powders, such as quartz, glass-ceramic, borosilicate or radiopaque glass powders, preferably barium or strontium aluminosilicate glasses, and radiopaque fillers, such as ytterbium trifluoride, tantalum(V) oxide, barium sulphate or mixed oxides of SiO₂ with ytterbium(III) oxide or tantalum(V) oxide. Furthermore, the dental materials according to the invention may contain fibrous fillers, nanofibers, whiskers or mixtures thereof. According to a preferred embodiment, the materials according to the invention do not contain fluoroaluminosilicate glasses, calcium aluminosilicate glasses or other fillers that react with organic acids in an acid-base reaction.

Preferably, the oxides have a particle size of 0.010 to 15 μm, the nanoparticulate or microfine fillers have a particle size of 10 to 300 nm, the glass powders have a particle size of 0.10 to 15 μm, preferably of 0.2 to 1.5 μm, and the radiopaque fillers have a particle size of 0.2 to 5 μm.

Particularly preferred fillers are mixed oxides of SiO₂ and ZrO₂, with a particle size of 10 to 300 nm, glass powders with a particle size of 0.2 to 1.5 μm, in particular X-ray opaque glass powders e.g. of barium or strontium aluminosilicate glasses, and X-ray opaque fillers with a particle size of 0.2 to 5 μm, in particular ytterbium trifluoride and/or mixed oxides of SiO₂ with ytterbium(III) oxide.

To improve the bond between the filler particles and the crosslinked polymerization matrix, SiO₂-based fillers can be surface-modified with methacrylate-functionalized silanes. A preferred example of such silanes is 3-methacryloyloxypropyltrimethoxysilane. The surfaces of non-silicate fillers such as ZrO₂ or TiO₂ can also be modified with functionalized acid phosphates, such as 10-methacryloyloxydecyl dihydrogen phosphate.

Other preferred fillers are particulate waxes, in particular carnauba wax, preferably with a particle size of 1 to 10 μm, non-crosslinked or partially crosslinked polymethyl methacrylate (PMMA) particles, preferably with a particle size of 500 nm to 10 μm, and polyamide-12 particles, preferably with a particle size of 5 to 10 μm. In addition, the dental materials according to the invention may contain a so-called prepolymer filler or isofiller, i.e. a ground composite which preferably has a broad particle size distribution, e.g. with particle sizes of 0.05 to 20 μm, in particular about 0.1 to about 10 μm. Preferably, the prepolymer filler or isofiller is surface modified, in particular salinized.

Unless otherwise stated, all particle sizes herein are weight-average particle sizes, and determination of particle sizes in the range of 0.1 μm to 1000 μm is performed by static light scattering, preferably using a static laser scattering particle size analyzer LA-960 (Horiba, Japan). Here, a laser diode with a wavelength of 655 nm and an LED with a wavelength of 405 nm are used as light sources. The use of two light sources with different wavelengths enables the measurement of the entire particle size distribution of a sample in only one measurement run, whereby the measurement is carried out as a wet measurement. For this, a 0.1 to 0.5% aqueous dispersion of the filler is prepared and its scattered light is measured in a flow cell. The scattered light analysis for calculating particle size and particle size distribution is carried out according to the Mie theory as per DIN/ISO 13320.

Particle sizes smaller than 0.1 μm are preferably determined by dynamic light scattering (DLS). The measurement of particle size in the range of 5 nm to 0.1 μm is preferably performed by Dynamic Light Scattering (DLS) of aqueous particle dispersions, preferably with a Malvern Zetasizer Nano ZS (Malvern Instruments, Malvern UK) with a He—Ne laser with a wavelength of 633 nm, at a scattering angle of 90° at 25° C.

The light scattering decreases with decreasing particle size. Particle sizes smaller than 0.1 μm can also be determined by SEM or TEM spectroscopy. Transmission electron microscopy (TEM) is preferably performed with a Philips CM30 TEM at an accelerating voltage of 300 kV. For sample preparation, drops of the particle dispersion are applied to a 50 Å thick copper grid (mesh size 300 mesh), which is coated with carbon, and the solvent is subsequently evaporated.

The fillers are subdivided according to their particle size into macrofillers and microfillers, whereby fillers with an average particle size of 0.2 to 10 μm are called macrofillers and fillers with an average particle size of about 5 to 100 nm are called microfillers. Macrofillers are obtained by grinding e.g. quartz, radiopaque glasses, borosilicates or ceramics and usually consist of splinter-shaped particles. Fumed SiO₂ or precipitated silica or mixed oxides, e.g. SiO₂—ZrO₂, which are accessible by hydrolytic co-condensation of metal alkoxides, are preferably used as microfillers. The microfillers preferably have an average particle size of about 5 to 100 nm. Fillers with a small particle size have a greater thickening effect.

In a preferred embodiment, the dental materials according to the invention contain a mixture of two or more fillers, in particular two or more fillers with different particle sizes. It has been found that the use of such filler mixtures does not excessively increase the viscosity of the materials and the compositions are therefore readily processable by generative methods, such as by stereolithography. The total content of fillers is preferably in a range from 0 to 30% by weight, particularly preferably from 0 to 20% by weight.

The compositions according to the invention may further contain one or more additives (h), in particular UV absorbers, optical brighteners, colorants, plasticizers and/or thixotropic additives.

The compositions according to the invention may contain one or more UV absorbers. UV absorbers serve to reduce the penetration depth of the light and thus the polymerization depth during the light-induced curing of the composition according to the invention. This proves to be particularly advantageous in stereolithographic applications, since only thin layers are to be cured in stereolithography. The use of a UV absorber can improve the precision of stereolithographic processes.

Preferred UV absorbers are those based on benzotriazole, benzophenone or triazines. Particularly preferred UV absorbers are 2,2′-methylenebis[6-(2H-benzotriazol-2-yl)-4-(1,1,3,3-tetramethylbutyl)phenol], 2,2′,4,4′-tetrahydroxybenzophenone, 2-tert-butyl-6-(5-chloro-2H-benzotriazol-2-yl)-4-methylphenol (bumetrizole), 2,2′-benzene-1,4-diylbis(4h-3,1-benzoxazin-4-one), 2-(4,6-bis-(2,4-dimethylphenyl)-1,3,5-triazin-2-yl)-5-(octyloxy)-phenol, 2-(2-hydroxy-5-methylphenyl)benzotriazole, 2-(2-hydroxyphenyl) benzotriazole, 2-(2H-benzotriazol-2-yl)-6-dodecyl-4-methylphenol, 2-(2′-hydroxy-3′,5′-di-t-butylphenyl)-5-chlorobenzotriazoles, 2,2′-dihydroxy-4-methoxybenzophenone and 2,2′-dihydroxy-4,4′-dimethoxybenzophenone. Further preferred are so-called hindered amine light stabilizers such as bis(1,2,2,6,6-pentamethyl-4-piperidyl)sebacate, methyl-i1,2,2,6,6-pentamethyl-4-piperidylsebacate, bis(1-octyloxy-2,2,6,6-tetramethyl-4-piperidyl)sebacate and bis(1,2,2,6,6-pentamethyl-4-piperidyl)-[[3,5-bis(1,1-dimethylethyl)-4-hydroxyphenyl] methyl]butylmalonate. Very particularly preferred UV absorbers are bumetrizole and 2,2′,4,4′-tetrahydroxybenzophenone.

The UV absorber preferably has an absorption maximum that corresponds to the wavelength of the light used for curing. UV absorbers with an absorption maximum in the range of 320 to 500 nm and preferably 380 to 480 nm are advantageous, whereby UV absorbers with an absorption maximum below 400 nm are particularly preferred.

UV absorbers are optionally used in an amount of preferably 0 to 1.0% by weight, more preferably 0.01 to 0.5% by weight. Bumetrizole is preferably used in an amount of 0.01 to 0.2% by weight, particularly preferably 0.02 to 0.15% by weight, and 2,2′,4,4′-tetrahydroxybenzophenone in an amount of 0.01 to 0.07% by weight. All data refer to the total weight of the material. Dental materials that do not contain a UV absorber are preferred.

The compositions according to the invention may further contain one or more optical brighteners. Preferred optical brighteners according to the invention are those that absorb light in the UV range, i.e. light with a wavelength below 400 nm. By adding an optical brightener, the penetration depth of the light and thus the curing depth can be reduced, thus increasing the precision in stereolithographic processes. Optical brighteners that are capable of re-emitting the light absorbed in the UV range as light with a wavelength of 400 to 450 nm are particularly preferred. Such optical brighteners increase the reactivity of the materials by emitting the absorbed short-wave light as longer-wave blue light due to their fluorescence, thus providing additional light power for photoinitiation. Optical brighteners preferred according to the invention are 2,5-bis(5-tert-butyl-benzoxazol-2-yl) thiophene and fluorescent agents in the form of terephthalic acid derivatives, such as 2,5-dihydroxyterephthalic acid diethyl ester or diethyl 2,5-dihydroxyterephthalate.

The optical brightener(s) may be used in an amount of preferably 0 to 0.1% by weight, more preferably 0.001 to 0.05% by weight and most preferably 0.002 to 0.02% by weight, each based on the total weight of the material. Dental materials that do not contain optical brighteners are preferred.

Optical brighteners can be used in combination with UV absorbers. In this case, it is preferred that the weight ratio of UV absorber to optical brightener is in the range of 2:1 to 50:1, more preferably 2:1 to 30:1 and most preferably 2:1 to 5:1 or 10:1 to 25:1. Preferred combinations are those containing 2,2′,4,4′-tetrahydroxybenzophenone or bumetrizole as UV absorbers and 2,5-bis(5-tert-butyl-benzoxazol-2-yl)thiophene as optical brightener. Very particularly preferred is the combination of 2,2′,4,4′-tetrahydroxy benzophenone and 2,5-bis(5-tert-butyl-benzoxazol-2-yl)thiophene in a weight ratio of 2:1 to 10:1, preferably 2:1 to 5:1, or the combination of bumetrizole and 2,5-bis(5-tert-butyl-benzoxazol-2-yl)thiophene in a weight ratio of 5:1 to 30:1, preferably 10:1 to 20:1.

The compositions according to the invention may further contain colorants, preferably in a concentration of 0.0001 to 0.5% by weight. The colorants primarily serve aesthetic purposes. Colorants preferred according to the invention are organic dyes and pigments, in particular azo dyes, carbonyl dyes, cyanine dyes, azomethines and methines, phthalocyanines and dioxazines. Particularly preferred are dyes which are soluble in the materials of the invention, especially azo dyes. Also suitable as colorants are inorganic and in particular organic pigments which can be readily dispersed in the dental materials according to the invention. Preferred inorganic pigments are metal oxides or hydroxides, such as titanium dioxide or ZnO as white pigments, iron oxide (Fe₂O₃) as red pigment or iron hydroxide (FeOOH) as yellow pigment. Preferred organic pigments are azo pigments, such as monoazo yellow and orange pigments, diazo pigments or p-naphthol pigments, and non-azo or polycyclic pigments, such as phthalocyanine, quinacridone, perylene and flavanthrone pigments. Azo pigments and non-azo pigments are particularly preferred.

In addition, the compositions according to the invention may contain one or more plasticizers. Plasticizers prevent the polymers from becoming brittle after photochemical curing and possible drying. In addition, plasticizers ensure sufficient flexibility. Plasticizers are preferably added in a concentration of 0.2 to 5% by weight. Preferred plasticizers are phthalates, such as dibutyl or dihexyl phthalate, non-acid phosphates, such as tributyl or tricresyl phosphate, n-octanol, glycerol or polyethylene glycols. Particularly preferred are tartaric or citric acid esters, such as citric acid triesters, which are characterized by good biocompatibility.

The compositions according to the invention may contain one or more thixotropic agents. These additives cause thickening of the materials and can thus, for example, prevent sedimentation of the fillers. Materials containing fillers in particular therefore preferably contain at least one thixotropic additive. Preferred thixotropy additives are polymers containing OH groups, such as cellulose derivatives, and inorganic substances, such as phyllosilicates. In order not to increase the viscosity of the materials too much, the dental materials according to the invention preferably contain only 0 to 3.0% by weight, more preferably 0 to 2.0% by weight and most preferably 0.1 to 2.0% by weight of thixotropic additive, based on the total weight of the material.

Certain fillers, such as highly dispersed SiO₂, i.e. SiO₂ with small primary particle size (<20 nm) and high specific surface area (>100 m²/g) also have a thixotropic effect. Such fillers can replace thixotropic additives.

The rheological properties of the compositions according to the invention are adapted to the desired application. Materials for stereolithographic processing are preferably adjusted so that their viscosity is in the range of 50 mPa·s to 100 Pa·s, preferably 100 mPa-s to 10 Pa·s, particularly preferably 100 mPa·s to 5 Pa·s. The viscosity is determined at 25° C. using a cone-plate viscometer (shear rate 100/s). Particularly preferably, the dental materials according to the invention have a viscosity <10 Pa·s and most preferably <5 Pa·s at 25° C. The viscosity is preferably determined with an Anton Paar viscometers of the type MCR 302 with CP25-2 cone-plate measuring means and a measuring gap of 53 μm in rotation at a shear rate of 100/s. Due to the low viscosity, the compositions according to the invention are particularly suitable to be processed by generative manufacturing methods, such as 3D printing or stereolithography. The processing temperature is preferably in a range from 10 to 70° C., particularly preferably 20 to 30° C.

The compositions according to the invention are particularly suitable as dental materials and in particular for the manufacture or repair of dental moldings, such as dental restorations, prostheses, prosthetic materials, artificial teeth, inlays, onlays, crowns, bridges, drilling templates, trial bodies or orthodontic devices. The use of the compositions according to the invention as dental materials is also an object of the invention.

Furthermore, the present invention also relates to a process for producing dental moldings, in particular for producing the above-mentioned dental moldings, in which a composition according to the invention is cured with the aid of light to yield the dental molding. Furthermore, the invention also relates to dental moldings, in particular the above-mentioned moldings, obtainable by such a process.

The fabrication or repair of dental moldings is preferably carried out extraorally. Furthermore, it is preferred that the production or repair of dental moldings is carried out by a generative process, in particular by means of 3D printing or a lithography-based process, such as stereolithography.

The production of dental moldings according to the invention is preferably carried out by a stereolithographic process. For this purpose, a virtual image of the tooth situation is created by directly or indirectly digitizing the tooth or teeth to be restored on the computer, then a model of the dental restoration or prosthesis is constructed on the computer on the basis of this image and this model is then produced by generative stereolithographic manufacturing. Once a virtual model of the dental restoration or prosthesis to be produced has been created, the composition according to the invention is polymerized by selective light irradiation. The geometry of the dental restoration or prosthesis can be built up layer by layer by successively polymerizing a plurality of thin layers with the desired cross-section. The layer-by-layer build-up of the geometry is usually followed by cleaning of the workpiece by treatment with a suitable solvent, e.g., an alcohol, such as ethanol or isopropanol, a ketone or an ester, and a post-treatment by irradiation of the workpiece with a suitable wavelength, e.g. an irradiation with an intensity of e.g. 25 mW/cm² at 405 nm and simultaneously 130 mW at 460 nm for 15 min. The workpiece is irradiated with light of suitable wavelength, optionally with simultaneous heating to 50° C. or more, in order to further reduce the residual monomer content and to improve the mechanical properties.

Post-treatment of free-radical polymers is understood to be a subsequent heat treatment and/or additional irradiation of the polymerizates to increase the double bond and monomer conversion and thus to improve the mechanical and optical properties of the polymers.

For post-treatment, it is advantageous to use two different initiators, e.g. two photoinitiators that differ in their absorption ranges, or one photoinitiator and one thermal initiator. Mixtures of photoinitiators for the UV range and the visible range are preferred.

The compositions according to the invention comprise at least one photoinitiator of formula (I). The photoinitiators of formula (I) belong to the UV photoinitiators, but they absorb up into the visible range, i.e. up to about 450 nm. Preferred UV photoinitiators for combination with the photoinitiators of formula (1) are acetophenones, e.g. 2,2-diethoxy-1-phenylethanone, benzoin ethers, such as Irgacure 651 (dimethyl benzilketal), hydroxyalkylphenylacetophenones, such as Irgacure 184 (1-hydroxy-cyclohexyl-phenyl-ketone), 2-benzyl-2-(dimethylamino)-4′-morpholinobutyrophenone (Irgacure 369) and 1-butanone-2-(dimethylamino)-2-(4-methylphenyl)-methyl-1-(4-morpholinyl)-phenyl (Irgacure 379).

The photoinitiators of formula (I) can also be combined with photoinitiators for the visible range. Suitable are a-diketones and their derivatives, such as 9,10-phenanthrenequinone, 1-phenyl-propane-1,2-dione, diacetyl or 4,4′-dichlorobenzil. Camphorquinone (CQ) and 2,2-dimethoxy-2-phenyl-acetophenone are preferably used, more preferably a-diketones in combination with amines as reducing agents, such as 4-(dimethylamino) benzoic acid ester (EDMAB), N,N-dimethylaminoethyl methacrylate, N,N-dimethyl sym.-xylidine or triethanolamine. Suitable monomolecular photoinitiators for the visible range are also monoacyltrialkyl-, diacyldialkyl- and tetraacylgermanium as well as tetraacylstannanes, such as benzoyl trimethyl germanium, dibenzoyl diethyl germanium, bis(4-methoxybenzoyl) diethyl germanium, tetrakis(2-methylbenzoyl) germanium or tetrakis(mesitoyl) stannane. Mixtures of the various photoinitiators can also be used, such as bis(4-methoxy benzoyl) diethylgermanium in combination with camphorquinone and 4-dimethylamino benzoic acid ethyl ester. However, according to the invention, compositions are preferred which, in addition to the photoinitiators of formula (I), do not contain any other photoinitiators which are active in the visible wavelength range. Particularly preferred are compositions which exclusively contain photoinitiators of formula (1) and no further photoinitiators, neither for the visible nor for the UV range.

Azo compounds, such as 2,2′-azobis-(isobutyronitrile) (AIBN) or azobis-(4-cyanovaleric acid), or peroxides, such as dibenzoyl peroxide, dilauroyl peroxide, tert-butyl peroctoate, tert-butyl perbenzoate or di-(tert-butyl) peroxide are preferred as additional thermal initiators. Combinations with aromatic amines can also be used to accelerate initiation by means of peroxides. Preferred redox systems are: Combinations of dibenzoyl peroxide with amines, such as N,N-dimethyl-p-toluidine, N,N-dihydroxyethyl-p-toluidine, p-dimethylaminobenzoic acid ethyl ester or structurally related systems.

The invention is explained in more detail below with reference to figures and examples.

FIG. 1 shows the change over time of the b* value according to the CIE-Lab color system of radically polymerizable compositions as a function of the storage time in water at 50° C. The compositions contain 1.0 wt. % of the photoinitiator TPO and different concentrations of the inhibitor MEHQ (curves from top to bottom 100, 200, 300, 500 ppm).

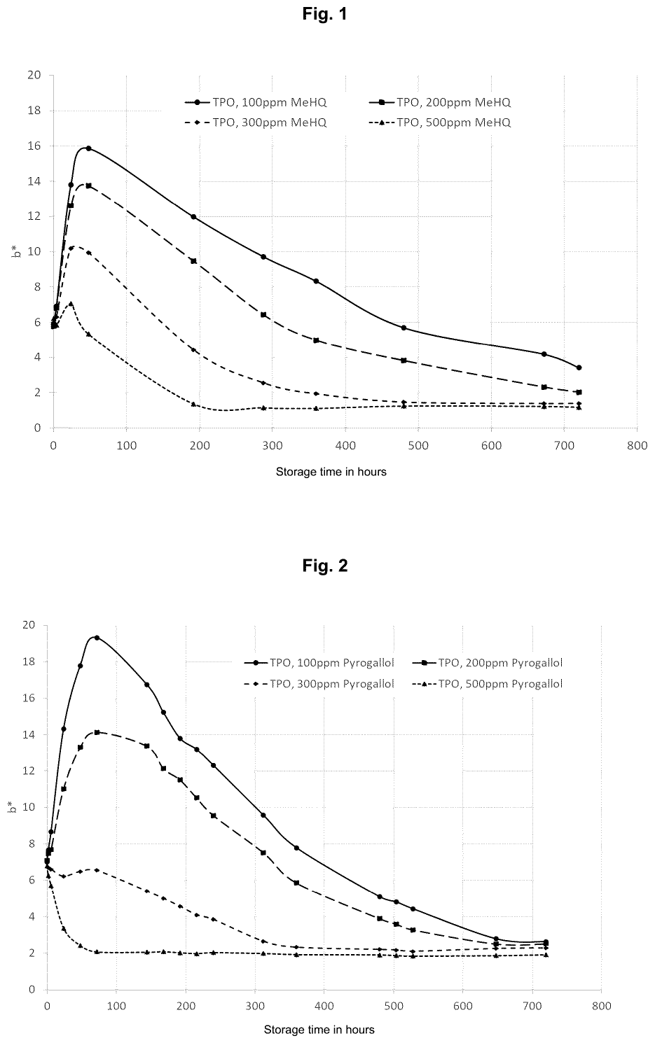

FIG. 2 shows the change over time of the b* value of compositions containing 1.0 wt. % of the photoinitiator TPO and different concentrations of the inhibitor pyrogallol (curves from top to bottom 100, 200, 300, 500 ppm).

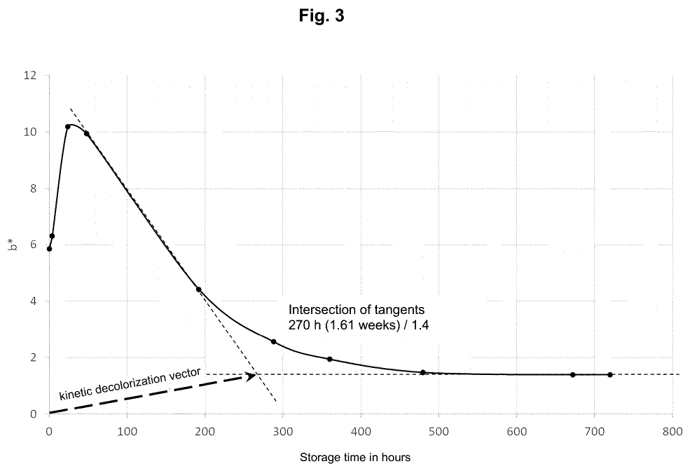

FIG. 3 illustrates the determination of the kinetic decolorization vector using the composition with 300 ppm of the inhibitor MEHQ from FIG. 1. The tangents to the falling flank and to the horizontal of the curve are shown. The straight line between the coordinate origin and the intersection of the tangents (arrow) corresponds to the kinetic decolorization vector.

### EXAMPLES

### Example 1

### Synthesis of (2,4-dimethoxy-6-methylbenzoyl)diphenylphosphine oxide (K-274)

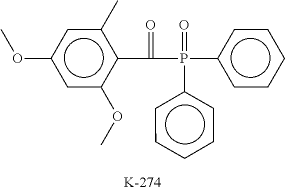

Triethylamine (0.79 g, 7.8 mmol) was slowly added dropwise to a mixture of 2,4-dimethoxy-6-methylbenzaldehyde (1.40 g, 7.8 mmol) and diphenylphosphine oxide (1.57 g, 7.8 mmol) in 50 mL THF. The reaction mixture was then stirred for 72 h at room temperature and the solvent was withdrawn. The yellowish oil that remained was then dissolved in 50 dichloromethane and activated manganese(IV) dioxide (17.00 g, 0.20 mol) was added. The suspension formed was then stirred at room temperature for 48 h, filtered over Celite and then the solvent was withdrawn. The crude product thus obtained was purified by column chromatography (SiO₂ column, eluent: ethyl acetate) and yielded 4.62 g (12.1 mmol; 44% yield) of a highly viscous yellowish oil. ¹H NMR (CDCl₃, 400 MHz): δ=7.89-7.80 (m, 4H; Ar—H), 7.55-7.42 (m, 6H; Ar—H), 6.35 (br s, 1 H; Ar—H), 6.30 (d, 1H; J=2.1 Hz; Ar—H), 3.79 (s, 3H; OCH₃), 3.54 (s, 3H; OCH₃), 2.15 (s, 3H; CH₃). ¹³C NMR (CDCl₃, 100.6 MHz): δ=211.1 (d, J=84 Hz; C═O), 163.8 (Ar—C), 162.0 (d, J=1 Hz; Ar—C), 140.4 (d, J=4 Hz; Ar—C), 131.6 (d, J=96 Hz; Ar—CH), 131.6 (d, J=3 Hz; Ar—CH), 131.5 (d, J=8 Hz; Ar—CH), 128.3 (d, J=11 Hz; Ar—CH), 120.4 (d, J=42 Hz; Ar—C), 109.1 (d, J=2 Hz; Ar—CH), 96.0 (Ar—CH), 55.6 (0-CH₃), 55.4 (0-CH₃), 19.9 (CH₃). ³¹P-NMR (CDCl₃, 162 MHz): δ=17.48.

### Example 2

### Synthesis of (2.4.6-trimethoxvbenzovl)diphenvlphosphine oxide (K-276)

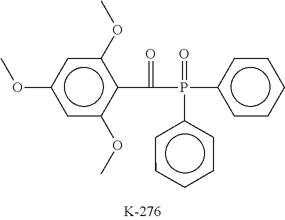

Analogous to Example 1, triethylamine (5.06 g, 50.0 mmol), 2,4,6-trimethoxybenzaldehyde (9.81 g, 50.0 mmol), and diphenylphosphine oxide (10.11 g, 50.0 mmol) were dissolved in 150 mL THE and reacted for 6 h at room temperature. After removal of the solvent the yellowish residue was taken up in 250 mL dichloromethane, activated manganese(IV) dioxide (86.94 g, 1.00 mol) was added and the suspension was stirred for 24 h at room temperature. After work-up analogous to Example 1 and column chromatography, 16.11 g (40.6 mmol; 81% yield) of the product were obtained as a yellowish solid.

¹H NMR (CDCl₃, 400 MHz): δ=7.88-7.79 (m, 4H; Ar—H), 7.53-7.38 (m, 6H; Ar—H), 6.07 (s, 2H; Ar—H), 3.78 (s, 3H; OCH₃), 3.60 (s, 6H; OCH₃). ¹³C NMR (CDCl₃, 100.6 MHz): δ=207.3 (d, J=88 Hz; C═O), 165.2 (Ar—C), 160.6 (Ar—C), 131.6 (d, J=96 Hz; Ar—CH), 131.5 (d, J=9 Hz; Ar—CH), 131.4 (d, J=2 Hz; Ar—CH), 128.1 (d, J=12 Hz; Ar—CH), 110.14 (d, J=44 Hz; Ar—C), 90.9 (Ar—CH), 55.6 (O—CH₃), 55.4 (O—CH₃). ³¹P-NMR (CDCl₃, 162 MHz): δ=17.18.

### Example 3

Preparation of polymerizable compositions with MEHQ The monomer UDMA (about 99 wt. %) was homogeneously mixed with 1 wt. % of one of the initiators listed in Table 1 each and the amounts of the inhibitor MEHQ given in Table 1. The components were dissolved in a planetary mixer with stirring, and stirring was continued until a homogeneous mixture was achieved. Completely transparent resins were always obtained. With the compositions from Table 1, test specimens were prepared in metal molds, which were irradiated on both sides for 2×1.5 minutes with a dental light source (PrograPrint Cure, Ivoclar Vivadent AG, Schaan, Liechtenstein; Software: ProArt Print Splint, Year: 2020) and thus cured. Completely transparent polymerizates were obtained.

To determine the color stability, the b* value of the samples was determined as a function of the storage time in water at 50° C. As an example, the results for the photoinitiator TPO and the stabilizer MEHQ are shown in FIG. 1. FIG. 1 shows that the monomer resins have an initial b* value of about 6. The b* value rises sharply at the start of polymerization and falls again during subsequent storage. The yellow value of the resin mixture M-1, which contains 100 ppm of the inhibitor MEHQ, increases from 6 to about 16. Only after storage for about 450 hours (about 19 days) does the yellow coloring return to its initial value of about 6. From a b* value of about 4.5, the yellow coloring is no longer perceptible to the human eye. This threshold is only reached after more than 600 hours (25 days). The mixtures M-3 (300 ppm MEHQ) and M-4 (500 ppm MEHQ), on the other hand, show not only a much smaller increase but also a much faster decrease in the yellow value. In both cases, the b* value falls below the perception threshold of 4.5 in less than 200 h (about 8 days).

To determine the kinetic decolorization vector of the resin mixtures, a tangent was applied to the falling flank and to the horizontal of the curve, as shown in FIG. 3. The coordinates of the intersection of the tangents (time and b* value) are given in Table 1. The straight line between the origin of the coordinates and the intersection of the tangents corresponds to the decolorization vector (arrow).

TABLE 1
Composition of materials with the inhibitor MEHQ

| | | | | Intersection of tangents | | | |
|---|---|---|---|---|---|---|---|
| Monomer | | | | | Time^{h)} | Time^{h)} | Vector |
| resin | Monomer | Photoinitiator^{b)} | MeHQ^{c)} | b* | [h] | [weeks] | Lengthⁱ⁾ |
| M-1 | UDMA^{a)} | TPO^{d)} | 100 | 1.6 | 670 | 3.99 | 4.30 |
| M-2 | UDMA | TPO | 200 | 1.6 | 460 | 2.74 | 3.17 |
| M-3 | UDMA | TPO | 300 | 1.4 | 270 | 1.61 | 2.13 |
| M-4 | UDMA | TPO | 500 | 1.1 | 205 | 1.22 | 1.64 |
| M-5 | UDMA | TPO-L^{e)} | 100 | 1.9 | 665 | 3.96 | 4.39 |
| M-6 | UDMA | TPO-L | 200 | 1.5 | 265 | 1.58 | 2.18 |
| M-7 | UDMA | TPO-L | 300 | 1.7 | 150 | 0.89 | 1.92 |
| M-8 | UDMA | TPO-L | 500 | 1.6 | 85 | 0.51 | 1.68 |
| M-9 | UDMA | K-274^{f)} | 100 | 5.8 | 685 | 4.08 | 7.09 |
| M-10 | UDMA | K-274 | 200 | 2.9 | 145 | 0.86 | 3.03 |
| M-11 | UDMA | K-274 | 300 | 1.9 | 110 | 0.65 | 2.01 |
| M-12 | UDMA | K-274 | 500 | 1.4 | 60 | 0.36 | 1.44 |
| M-13 | UDMA | K-276^{g)} | 100 | 3.8 | 410 | 2.44 | 4.52 |
| M-14 | UDMA | K-276 | 200 | 2.7 | 150 | 0.89 | 2.84 |
| M-15 | UDMA | K-276 | 300 | 2.5 | 90 | 0.54 | 2.56 |
| M-16 | UDMA | K-276 | 500 | 2.1 | 75 | 0.45 | 2.15 |

^{a)}UDMA: an addition product of 2-hydroxyethyl methacrylate and 2,2,4-trimethylhexamethylene-1,6-diisocyanate, amount = difference to 100 wt. %
^{b)}Photoinitiator quantity: 1 wt. %
^{c)}MEHQ: hydroquinone monomethyl ether, quantity given in ppm
^{d)}TPO: diphenyl-(2,4,6-trimethylbenzoyl)phosphine oxide (Sigma-Aldrich)
^{e)}TPO-L: 2,4,6-trimethylbenzoylethoxylphenylphosphine oxide (IGM Resins)
^{f)}K-274: (2,4-dimethoxy-6-methylbenzoyl)diphenylphosphine oxide from Ex. 1
^{g)}K-276: (2,4,6-Trimethoxybenzoyl)diphenylphosphine oxide from Ex. 2
^{h)}Time: Storage time of the sample at 50° C.
ⁱ⁾value (=length) of the kinetic decolorization vector, calculated as \sqrt{(b*)² + (time [weeks])²}

### Example 4

Preparation of Polymerizable Compositions with Pyrogallol

The components listed in Table 2 were homogeneously mixed together in the same way as in Example 3 and test specimens were produced from the mixtures. These were completely transparent. The initiator concentration was 1 wt. % in each case. The inhibitor pyrogallol was used in the amounts listed in Table 2. The amount of the monomer UDMA was about 99 wt. % in each case.

To determine the color stability, the b* value of the samples was determined as a function of the storage time in water at 50° C. As an example, the results for the photoinitiator TPO and the stabilizer pyrogallol are shown in FIG. 2. The mixture M-19 (300 ppm pyrogallol) shows only a small increase of the b* value during polymerization, while the b* value of the sample M-20 (500 ppm pyrogallol) does not show any increase, but drops immediately after the start of polymerization. In both cases, the b* value falls below the perception threshold of 4.5 in less than 200 h (about 8 days). Sample M-20 reaches this value already after about 25 h.

The determination of the kinetic decolorization vector was carried out analogously to example 3. The results are given in table 2.

TABLE 2
Composition of the materials with the inhibitor pyrogallol

| | | | | Intersection of tangents | | | |
|---|---|---|---|---|---|---|---|
| Monomer | | | | | Time^{h)} | Time^{h)} | Vector |
| resin | Monomer | Photoinitiator^{b)} | PGL^{c)} | b* | [h] | [weeks] | Lengthⁱ⁾ |
| M-17 | UDMA^{a)} | TPO^{d)} | 100 | 2.6 | 495 | 2.95 | 3.93 |
| M-18 | UDMA | TPO | 200 | 2.3 | 385 | 2.29 | 3.25 |
| M-19 | UDMA | TPO | 300 | 2.2 | 5 | 0.03 | 2.20 |
| M-20 | UDMA | TPO | 500 | 2.3 | 5 | 0.03 | 2.30 |
| M-21 | UDMA | TPO-L^{e)} | 100 | 2.7 | 485 | 2.89 | 3.95 |
| M-22 | UDMA | TPO-L | 200 | 2.6 | 462 | 2.75 | 3.78 |
| M-23 | UDMA | TPO-L | 300 | 2.4 | 330 | 1.96 | 3.10 |
| M-24 | UDMA | TPO-L | 500 | 2.1 | 32 | 0.19 | 2.11 |
| M-25 | UDMA | K-276^{g)} | 100 | 6.4 | 420 | 2.50 | 6.87 |
| M-26 | UDMA | K-276 | 200 | 4.9 | 400 | 2.38 | 5.45 |
| M-27 | UDMA | K-276 | 300 | 3.3 | 33 | 0.20 | 3.31 |
| M-28 | UDMA | K-276 | 500 | 3.3 | 30 | 0.18 | 3.30 |

^{a)}UDMA: an addition product of 2-hydroxyethyl methacrylate and 2,2,4-trimethylhexamethylene-1,6-diisocyanate
^{b)}Photoinitiator quantity: 1 wt. %
^{c)}PGL: pyrogallol, quantity given in ppm
^{d)}TPO: diphenyl-(2,4,6-trimethylbenzoyl)phosphine oxide (Sigma-Aldrich)
^{e)}TPO-L: 2,4,6-trimethylbenzoylethoxylphenylphosphine oxide (IGM Resins)
^{f)}K-276: (2,4,6-trimethoxybenzoyl)diphenylphosphine oxide from Ex. 2
^{g)}Time: Storage time of the sample at 50° C.
^{h)}value (=length) of the kinetic decolorization vector, calculated as \sqrt{(b*)² + (time [weeks])²}

1. An initiator system for radical polymerization, comprising at least one monoacylphosphine oxide of formula (1) 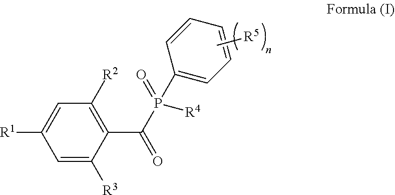
in which
R¹, R², R³ independently or one another are each H, or a linear or branched C₁-C₁₀ alkyl radical or OR⁶,
R⁴ is a linear or branched C₁-C₁₀ alkyl radical, C₁-C₁₀ alkoxy radical, cyclohexyl radical or a phenyl, methylphenyl or mesityl radical,
R⁵ is a linear or branched C₁-C₁₀ alkyl radical,
R⁶ is a linear or branched C₁-C₆ alkyl radical, and
n is 0, 1, 2 or 3,
and at least one phenolic inhibitor, characterized in that the molar ratio of inhibitor to photoinitiator is in a range from 0.002 to 0.9.

2. The initiator system according to claim 1, wherein the variables have the following meanings:
R¹,R², R³ independently of one another are each a linear or branched C₁-C₃ alkyl radical or OR⁶,
R⁴ is a linear or branched C₁-C₅ alkyl, C₁-C₅ alkoxy, cyclohexyl or phenyl radical,
R⁵ is a linear or branched C₁-C₃ alkyl radical,
R⁶ is a linear or branched C₁-C₃ alkyl radical, and
n is 0, 1 or 3.

3. The initiator system according to claim 1, wherein the variables have the following meanings:
R¹, R², R³ independently of one another are each H, a linear or branched C₁-C₃ alkyl radical or OR⁶,
R⁴ is a linear or branched C₁-C₅ alkyl, C₁-C₅ alkoxy, cyclohexyl or phenyl radical,
R⁵ is a linear or branched C₁-C₃ alkyl radical,
R⁶ is a linear or branched C₁-C₃ alkyl radical, and
n is 0, 1, 2 or 3,
wherein if R⁴ is not a phenyl radical, n is 0, and if R⁴ is a phenyl radical, n is 1, 2 or 3.

4. The initiator system according to claim 1, wherein the variables have the following meanings:
R¹ is H, a linear or branched C₁-C₃ alkyl radical or OR⁶,
R², R³ independently of one another are each H, a linear or branched C₁-C₃ alkyl radical or OR⁶,
R⁴ is a linear or branched C₁-C₅ alkyl, C₁-C₅ alkoxy, cyclohexyl or phenyl radical,
R⁵ is a linear or branched C₁-C₃ alkyl radical,
R⁶ is a linear or branched C₁-C₃ alkyl radical,and
n is 0, 1 or 3.

5. The initiator system according to claim 1, wherein at least one of R¹, R² and R³ is OR⁶.

6. The initiator system according to claim 1, wherein the phenolic inhibitor (f) is selected from hydroquinone monomethyl ether, 2,6-di-tert-butyl-4-methyl phenol, 2,6-di-tert-butyl phenol, pyrogallol, hydroquinone, 2-tert-butyl hydroquinone, 4-tert-butylcatechol, 6-tert-butyl-2,4-xylenol, or a mixture thereof.

7. A radically polymerizable composition, comprising an initiator system according to claim 1 and at least one urethane group-containing (meth)acrylate monomer.

8. The composition according to claim 7, comprising
(a) 0.1 to 8% by weight of the at least one monoacylphosphine oxide of formula (1),
(b) 1 to 99% by weight of the at least one urethane group-containing (meth)acrylate monomer,
(c) 0 to 70% by weight of one or more aliphatic, cycloaliphatic, aromatic, bicyclic or tricyclic mono(meth)acrylates,
(d) 0 to 50% by weight of one or more di(meth)acrylates without urethane groups,
(e) 0 to 12% by weight of one or more block copolymers and/or core-shell polymers,
(f) 200 to 1200 ppm of the at least one phenolic inhibitor,
(g) 0 to 50% by weight of one or more inorganic and/or organic fillers,
(h) 0 to 30% by weight of further additives,
in each case in relation to the total mass of the composition.

9. The composition according to claim 8, comprising
(a) 0.30 to 6.5% by weight of at least one of the monoacylphosphine oxide of formula (I),
(b) 1 to 80% by weight of at least one of the urethane group-containing monomer,
(c) 0 to 66% by weight of one or more of the aliphatic, cycloaliphatic, aromatic, bicyclic or tricyclic mono(meth)acrylates,
(d) 0 to 40% by weight of one or more of the di(meth)acrylates without urethane groups,
(e) 0 to 10% by weight of one or more of the block copolymers and/or core-shell polymers,
(f) 300 to 1000 ppm of the at least one phenolic inhibitor,
(g) 0 to 40% by weight of one or more of the inorganic and/or organic fillers,
(h) 0 to 25% by weight of one or more of the further additives selected from UV absorbers, optical brighteners, colorants, plasticizers and thixotropic additives.

10. The composition according to claim 7, wherein the urethane group-containing (meth)acrylate monomer is a urethane di(meth)acrylate.

11. The composition according to claim 7, which has a kinetic decolorization vector of less than 3.5, wherein the decolorization vector is determined in the manner described in the description.

12. A process for the production of dental molded bodies, comprising
(i) creating a virtual image of a tooth situation by directly or indirectly digitizing the tooth or teeth to be restored on the computer,
(ii) constructing a model of a dental restoration or prosthesis on the computer using the virtual image,
(iii) building up the dental restoration or prosthesis layer by layer by polymerization of a composition according to claim 7 by selective light irradiation.

13. The method according to claim 12, wherein the dental molded bodies have a b* value below 4.5 according to the CIE Lab color system.
